(19) United States
(12) Patent Application Publication
(10) Pub. No.: US 2025/0073104 A1
(43) Pub. Date: Mar. 6, 2025

(54) INDIVIDUAL PRESSURE ZONE CONTROLLED CUSHION AND SUPPORT

(71) Applicant: KALOGON, INC., MELBOURNE, FL (US)

(72) Inventors: Timothy Balz, Melbourne, FL (US); Christian Balcom, Cocoa, FL (US); Connor Crenshaw, Orlando, FL (US); Aaron Jones, Melbourne, FL (US); Evan Rosenberg, Rockledge, FL (US)

(21) Appl. No.: 18/579,666

(22) PCT Filed: Jul. 16, 2022
(86) PCT No.: PCT/US2022/037404

§ 371 Date: Jan. 16, 2024

(30) Foreign Application Priority Data
Jul. 17, 2021 (US) 63222955
Jul. 16, 2022 (US) 17866491

(51) Int. Cl. A61G 7/057; G16H 40/63

(52) U.S. Cl. CPC A61G 7/05776; G16H 40/63; A61G 2203/34

(57) ABSTRACT

A cushion promotes healthy blood flow within the body when the patient is sitting/lying down. A pressure controlling support automatically balances user weight, detects application of user weight, or otherwise detects when the user is at-risk for pressure sores. Pressure is dynamically redistributed by inflation/deflation of individual bladders, rebalancing user support. The cushion prevents the user from applying their weight to impede their blood flow at an at-risk location or from cramping their musculature. The cushion also massages the user's musculature, by moving a pressure difference about selected bladders, promoting blood flow and reducing seating fatigue. The cushion detects changes in pressure at each bladder, to detect/predict user fidgeting/movement and adjust pressure in response thereto, to support the user's new position.

### BACKGROUND

This Application describes technologies that can be used with inventions, and other technologies, described in a Technical Appendix, hereby incorporated by reference as if fully set forth herein.

Patients and other users who have restricted mobility, or who otherwise have difficulty obtaining comfortable seating, might sometimes be subject to adverse medical conditions, such as pressure sores and/or decubitus ulcers. These conditions, left undetected or untreated, can involve serious adverse consequences, such as loss of muscle mass, serious infection, and even death.

Accordingly, it would be advantageous to provide a device that can be responsive to such patients, is relatively accommodating to user needs or preferences, and is relatively inexpensive in comparison with known devices.

The technologies described herein can also be useful for patients and other users, whether they have restricted mobility or are otherwise fully mobile. When users remain seated, or are otherwise restrained, for an extended period of time (such as being confined to a hospital bed or to a wheelchair), those users might be subject to “seating fatigue”. This can lead to camped muscles, pain, sores, or other discomfort, even for completely healthy persons. For example, persons who participate in extended automobile or airline travel might find it necessary to leave their seats and stretch periodically. But this can be difficult in a moving vehicle or if the user is otherwise temporarily constrained.

Accordingly, it would be advantageous to provide a device that can be responsive to those patients and other users, such as to allow for more extended seating without discomfort and without the user's desire for breaks.

### SUMMARY OF THE DISCLOSURE

This Application describes devices, and methods for using them, that are disposed to promote healthy blood flow within the body, such as the buttock and leg region when the patient is sitting, or the back region when the patient is lying down or leaning on a support. This can include a pressure controlling cushion and/or support that can automatically balance or offset the user's weight, whether the user continuously and improperly applies their weight to a fixed location, or whether the user applies their weight to an at-risk location, or otherwise as described herein. Pressure can be distributed or redistributed, by inflation/deflation of individual bladders, to dynamically balance or rebalance pressure underneath the user. Thus, if the user applies their weight to a fixed location or an at-risk location, the cushion/support can prevent that weight from impeding the user's blood flow, cramping the user's musculature, or otherwise damaging the user.

The cushion and/or support can also massage the user's musculature to promote blood flow and to reduce seating fatigue. The cushion can select an amount of pressure which is more or less than an amount used for support of the user (thus, a pressure difference). The cushion can select individual bladders to which to apply the pressure difference, thus causing the user to perceive that the pressure difference is effectively massaging the user's musculature. This can have a positive effect on the user's musculature, similar to when the user fidgets or shifts in their seat or gets up and stretches.

The cushion and/or support can reduce pressure load on the user's load bearing points, such as when the user is in a sitting and/or lying down position. This can assist individuals in attaining comfortable seating, in avoiding pressure sores or associated medical conditions, in improving circulation, and in otherwise improving the user's seating comfort. The cushion and/or support can be disposed to aid individuals in the recovery process of healing from a decubitus ulcer, or the process of healing from related medical conditions sometimes resulting from relative immobility. The cushion and/or support can also be disposed to aid otherwise mobile individuals who must remain seated or otherwise relatively motionless for an extended period of time. This can have a positive effect of reducing pain, cramping, or stiffness, when the user maintains substantially the same position for an extended time.

The cushion and/or support can detect changes in amount of pressure applied to each bladder. Thus, when the user fidgets or shifts in their seat, the cushion can detect that movement and measure how often and how extensive is that movement. In one embodiment, the cushion and/or support (or a controller coupled thereto) can predict user movement and adjust the amount of pressure in response to predicted movement. Thus, when the user moves, the cushion and/or support can adjust the pressure associated with one or more bladders to support the user in their new position. If the user does not move for an extended period of time, the cushion and/or support can adjust the pressure to massage the user's musculature, to prevent pressure sores or other adverse medical conditions. If the user gets up from, or sits/lies down on, the cushion and/or support, it can detect this condition and turn on/off in response thereto.

In one embodiment, the device can be disposed to inflate/deflate regions, such as using inflatable/deflatable bladders, supporting the patient, so as to provide (A) therapeutic massaging of affected regions and regions that are subject to possible pressure sores or related medical conditions, (B) comfortable support to muscles and other regions subject to possible pressure sores or subject to pressure from internal bony structures, (C) detection of patient movement, or lack thereof, possibly leading to patient injury, such as from pressure sores or from falling or sliding off the support, (D) detection of inadequate blood flow or other initial signs of pressure sores, possibly leading to patient injury, such as from unhealthy blood flow, muscular pinching, or otherwise as described herein.

### User Information

In one embodiment, the device can be disposed to receive, maintain, and process, information from the patient with respect to the user's (patient's) sitting (or lying down) behavior. This can have the effect that the devices can be disposed to determine a model of user behavior and one or more optimal methods of treatment suited to the patient. For example, the device can be disposed to determine one or more methods for therapeutic massaging of the patient's body, including determining which methods are best suited to the patient when the patient is relatively active or relatively immobile.

In one embodiment, the device can be disposed to receive information from the patient with respect to pain or related medical conditions. For example, the device can be disposed to respond to the patient's indication of pain with one or more changes in static pressure, or in dynamic pressure adjustments, so as to improve patient comfort and/or to reduce the likelihood of pressure sores or other debilitating medical conditions. For another example, the patient's degree of comfort or discomfort can be inferred by the device in response to a measure of movement levels (or lack thereof) by the patient. For another example, the device can be disposed to respond to the patient's indication of pain with one or more determinations of a severity of the patient's medical condition. For another example, the device can be disposed to receive information from medical personnel, and act on that information, with respect to the patient's medical condition.

In one embodiment, the device can be disposed to alert the patient and/or caregivers that the patient (A) is, or is about to, fall or slide off a support, such as when the patient loses their balance or grip and slides off a seat, (B) is failing to move sufficiently so as to avoid pressure sores or pain, such as when the patient is unable to move about while on a support, such as when the patient has insufficient muscle tone or muscle control, or (C) is otherwise unable to avoid pressure sores or pain and needs assistance, such as from a caregiver. For example, the alert can include an audible or visual alarm, or can include an electronic signal to an external device that is disposed to alert the patient or a caregiver.

In one embodiment, the device can be disposed to automatically turn on in response to the patient sitting down or lying down on the device. Similarly, the device can be disposed to automatically turn off in response to the patient getting up from the device and no longer placing any pressure thereon. This can have the effect that the device can be left in a substantially off state while the patient is not using it, thus using a relatively less amount of power and not degrading the useful life of device components while not in use. This can also have the effect that the device can notify one or more caretakers when the patient lifts themselves up from the cushion, or otherwise gets up from the device.

In one embodiment, the device can be disposed to encourage the patient to lift themselves completely up from the device, thus reducing pressure on the device to zero (or nearly zero); this is sometimes referred to as an “offload exercise”. For example, the device can be disposed to alert the patient and one or more caretakers if the patient has not performed an offload exercise in a selected amount of time. In such cases, the device can include a speaker capable of notifying the patient, preferably in a pleasant manner, to improve their medical condition, such as through better compliance with offload exercises and/or other medical advice.

In one embodiment, the device can be disposed to respond to a user interface in addition to or in lieu of its automatic response to user fidgeting or movement (or lack thereof). For example, the device can be disposed to respond to a user interface on a set of buttons, a touch panel, one or more voice commands, or otherwise as described herein. Thus, the user can configure the device using the user interface to suit the user's individual preferences. The user interface can be wirelessly coupled to the device from a remote control physically separate but still reachable by the user. Moreover, the user interface can be disposed to calibrate and adjust the device so as to account for differences in user size, weight, and firmness preference. The device can also be disposed to determine a map of pressure on the cushion when the user is in a relatively neutral position; this map can be used to calibrate the cushion for the user, and to calibrate control with respect to pressure on specific locations, either generally or specific to the particular user.

### Pressure Management

In one embodiment, the pressure on each bladder supporting a patient (such as one supporting a region underneath the patient) can be measured concurrently with inflation/deflation of those bladders, notwithstanding that inflation/deflation and pressure affect each other and produce substantially inaccurate concurrent measurements. A software controller can be disposed to inflate/deflate those bladders and to concurrently measure pressure, such as by predicting an actual pressure in response to a measured pressure while inflation/deflation is being performed. Moreover, the device can distinguish a pressure measured for each bladder due to a weight of the patient from a pressure measured for each bladder due to internal gas pressure. The software controller can be disposed to determine the separable amounts of pressure, such as in response to a Kalman filter applied to a set of (possibly unreliable) measurements. This can have the effect that no separate measurement of the patient's location or weight, such as using strain gauges, is involved.

In one embodiment, the device includes deflation apparatus, such as shown with respect to FIG. 12, the exit orifice of the pneumatic system, such as shown with respect to the Pneumatic Diagram, can be routed back into the cushion component. This can have the effect that air vented from the bladders will exit directly into the cushion component and provide active cooling to areas where the user might be warming the cushion component by their natural body heat. For example, an additional hose (such as a 6th hose when there are 5 air bladders) can be routed through the hose sheath, such as shown with respect to the figures relating to the cushion component and its air bladders, so as to provide venting into and around the foam layer of the cushion component and reduce excessive heat and moisture buildup experienced by the user. For another example, when the system is coupled to an external source (either an external power source or an external pumping source), air can be pumped relatively continuously through the vent port so as to cool the cushion, as battery life or power are not likely to be a limitation.

### Cushion Interface

In one embodiment, the pneumatic solenoid valves and pump mountings can be disposed to mitigate sounds experienced by the user that might be produced by either the positive displacement pump (such as described with respect to FIG. 10) and normally closed pneumatic solenoid valves (such as described with respect to FIG. 9). For example, the valves or mountings can be suspended on rubber housings (such as described with respect to FIG. 10, element 004, or FIG. 9, element 001) to couple the hardware without necessarily rigidly connecting it to the electrical subsystem or its housing. Additionally, foam can be wrapped around the pumps and solenoids, or otherwise disposed so as to dampen the sound and vibrations.

In one embodiment, the pressure cushion system can include a quick-disconnect interface between the cushion component and the pneumatic subsystem. In one embodiment, the quick disconnect interface can include a set of quick-disconnect couplings, such as a set of pneumatic snap-on quick disconnects (such as shown with respect to FIG. 6). The snap-on quick disconnects can be coupled to a fused deposition modeling (FDM) 3D printed housing made of polyethylene terephthalate (PETG), or another suitable material, that arranges those disconnects in an asymmetric pattern (also such as shown with respect to FIG. 6). Alternatively, the housing can be constructed using injection molding or another suitable technique. This can have the effect of ensuring that the user can only couple the cushion component to the control housing in one predetermined orientation. This can have the effect of providing modularity, allowing the cushion component to be altered, customized, or otherwise enhanced, independently of the controller component; this can have the advantage that distinct support surfaces of the cushion component can be used or repaired, modified, cleaned, or otherwise adjusted as described herein, without involving corresponding changes to the controller component. The asymmetric pattern can include a single selected (such as standardized) manifold; this can have the effect of simplifying the coupling between the cushion component and the pneumatic subsystem into a substantially single part. Alternatively, the system can be disposed to include an external interface having a male/female (or other asymmetric) connector, so as to provide that when the coupling is incorrect, the male/female connector does not fit.

In one embodiment, the pressure cushion system can include a user interface, such as a set of touch controls or a program app executable on a smartphone or other mobile device or a console or other wired controller, disposed to provide the user with the ability to control operation of the system. For example, the user interface can provide a display of a pressure map, showing where the user is exerting pressure (or undue pressure, if requested) on the cushion component. For another example, the user interface can provide an alert to the user or to one or more caretakers if the user is about to slide off the cushion component. For another example, the user interface can provide an alert to the user or to one or more caretakers if the user has remained substantially immobile or otherwise is at risk for a medical problem.

In one embodiment, the user interface can be coupled to the pressure cushion system using a wireless connection, such as using a wireless capability of a smartphone or another mobile device. Similarly, the pressure cushion system can couple at least a portion of the cushion component (such as the air bladders, pumping or venting elements, or solenoids coupled thereto) to at least a control portion of the electrical subsystem using a wireless connection.

### Electrical Subsystem

In one embodiment, the electrical subsystem can be disposed to halt an inflate/deflate operation in response to an error determination, for example, so as to prevent or mitigate overinflation or underinflation. In such cases, pressure data from the bladders may be coupled to the electrical subsystem so as to determine whether one or more bladders are not maintaining pressure within a set of selected limits.

In one embodiment, an additional pressure transducer may be coupled to one or more selected pump or vent lines, so as to allow for fault tolerance. For example, when pressure is added or removed from selected bladders, the value on the additional pressure transducer can be disposed to be compared with a dedicated transducer for the respective bladder. If the difference between the transducers is greater than a selected threshold, the additional pressure transducer can be checked against another selected one of the dedicated transducers; the malfunctioning transducer can be determined; and appropriate action can be taken. A relief valve can also be coupled to one or more selected pump or vent lines, or to one or more selected bladders, so that if overinflation occurs (or is threatened to occur), pressure can be vented to ameliorate the problem.

### BRIEF DESCRIPTION OF THE FIGURES

FIG. 1 shows a conceptual drawing of an example pressure cushion suitable for a patient to sit or rest upon, including at least elements shown in the figure.

FIG. 2 shows a conceptual drawing of an example internal structure of a pressure cushion, including at least elements shown in the figure.

FIG. 3 shows a conceptual drawing of an example inflatable air bladder disposed to support a patient on the pressure cushion, including at least elements shown in the figure.

FIG. 4 shows a conceptual drawing of an example set of regions that can be inflated/deflated in response to patient movement, including at least elements shown in the figure.

FIG. 5 shows a conceptual drawing of an example connection component between a pressure cushion and its control sensors and pumps, including at least elements shown in the figure.

FIG. 6 shows a conceptual drawing of an example set of connectors on a pressure cushion, including at least elements shown in the figure.

FIG. 7 shows a conceptual drawing of an example pump and mounting assembly, including at least elements shown in the figure.

FIG. 8 shows a conceptual drawing of an example controller box, including at least elements shown in the figure.

FIG. 9 shows a conceptual drawing of an example detailed view of a controller box, including at least elements shown in the figure.

FIG. 10 shows a conceptual drawing of an example housing and air compressor/pump, including at least elements shown in the figure.

FIG. 11 shows a conceptual drawing of an example set of mounting tabs for hoses coupling an air compressor/pump and a pressure cushion, including at least elements shown in the figure.

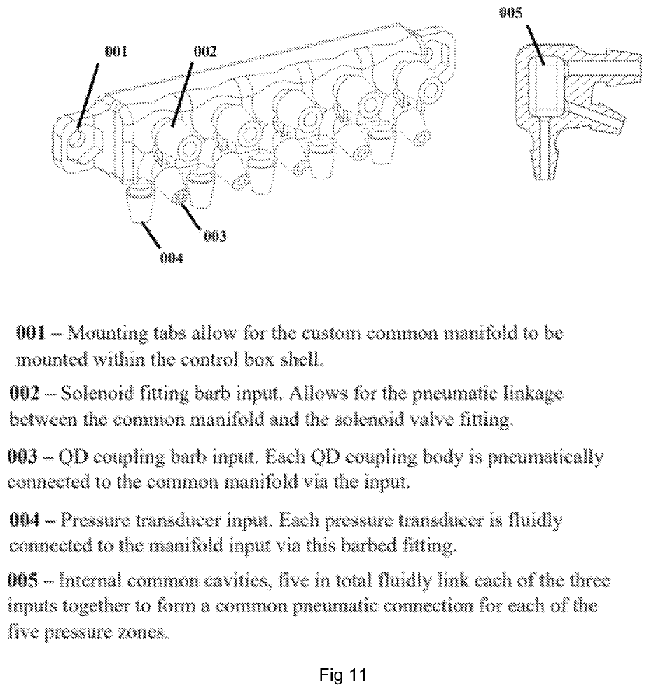

FIG. 12 shows a conceptual drawing of an example diagram of a set of controllers and valves for inflation/deflation of portions of the pressure cushion, including at least elements shown in the figure.

FIG. 13 shows a conceptual drawing of an example electrical block diagram of pressure cushion control unit, including at least elements shown in the figure.

FIG. 14 shows a conceptual drawing of an example schematic diagram of an analog front end for the device, including at least elements shown in the figure.

FIG. 15 shows a conceptual drawing of an example schematic diagram of a valve and pump driver, including at least elements shown in the figure.

FIG. 16 shows a conceptual drawing of an example power management system, including at least elements shown in the figure.

### DETAILED DESCRIPTION

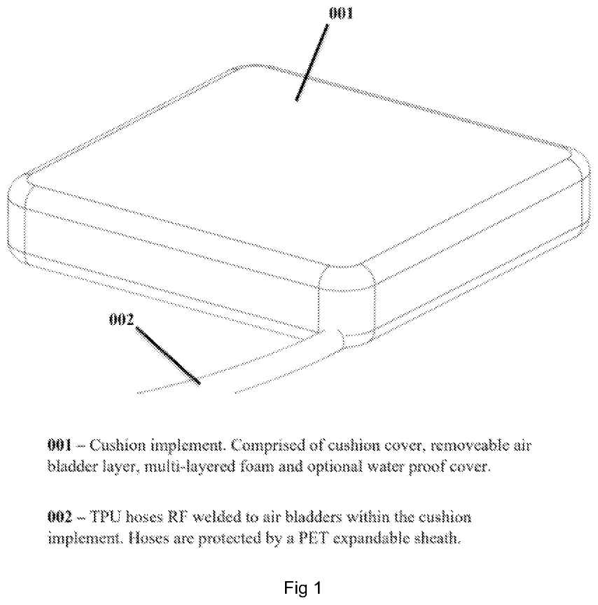

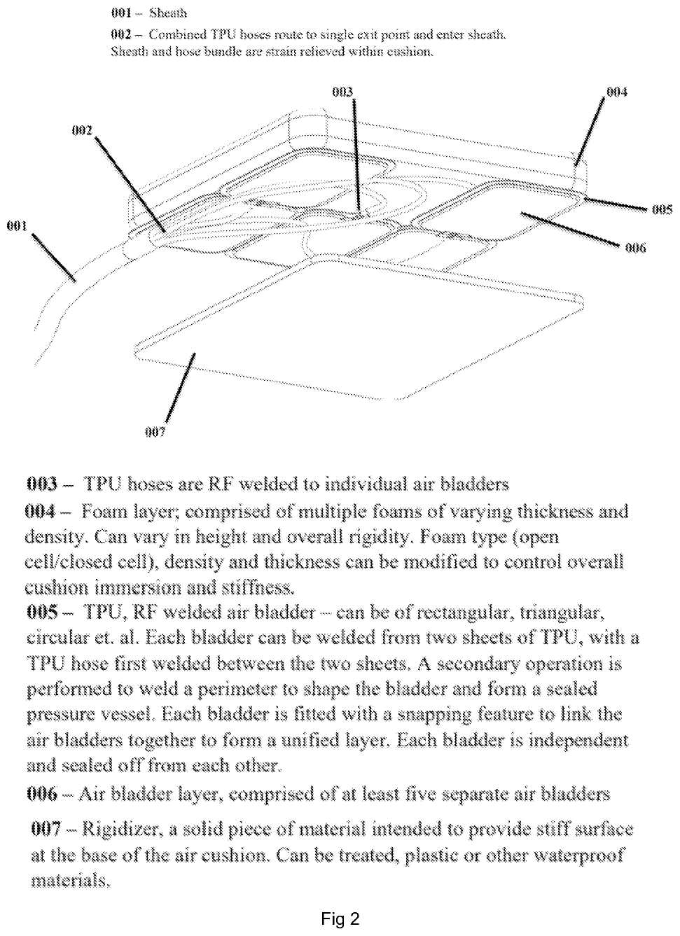

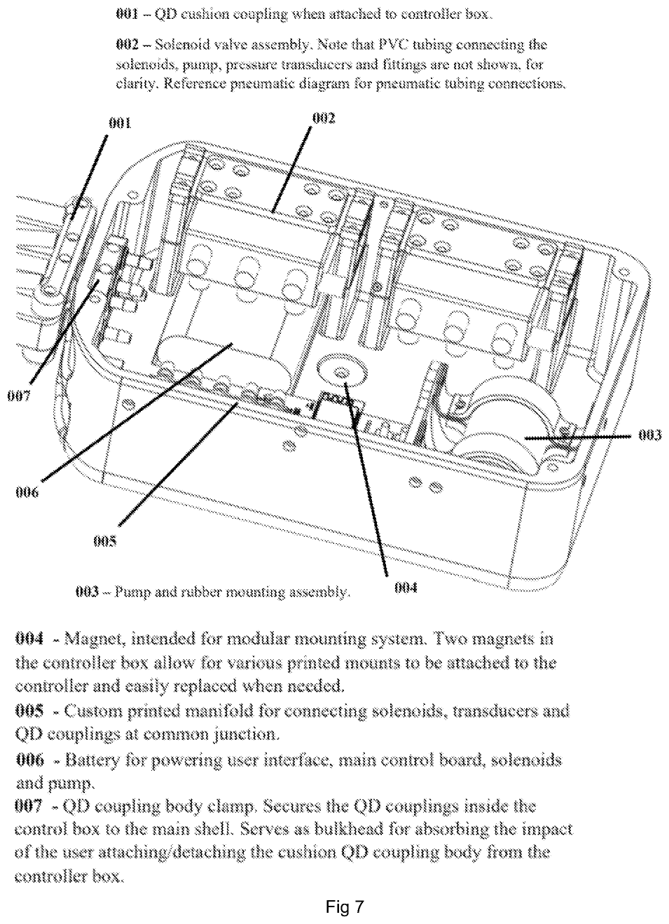

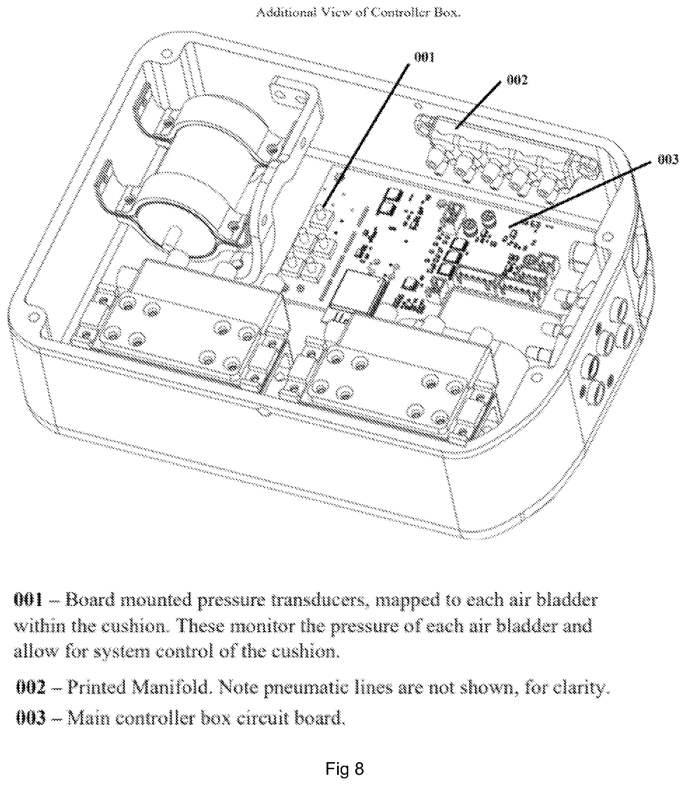

In one embodiment, a pressure cushion system includes (A) a cushion component, such as shown and described at least with respect to FIGS. 1-2, and (B) a control system component, such as shown and described at least with respect to FIGS. 7-8.

### Cushion Component

In one embodiment, the cushion component includes
A fabric sheet or another fabric-like substance. The fabric sheet is suitable to protect other elements of the cushion component from spills or other interference with the cushion component. The fabric sheet is preferably disposed to surround the entire element upon which the user can sit or lay.
A foam element or another padding material, possibly including a set of foam layers or other padding layers, disposed below the fabric sheet. The foam element is suitable for supporting the patient without direct contact with the air bladder layer, and for protecting the patient from any hard elements of the air bladder layer.
An air bladder layer, suitable for supporting a patient to sit or lay upon, and including a set of air bladders that can be inflated/deflated to support the patient, disposed below the foam element. The air bladders are disposed to be coupled to the control system, using a set of flexible tubing, such as using hoses disposed to withstand high pressure. As shown in the figures, the air bladder layer can include 5 air bladders (or another number of air bladders) so as to separately inflate/deflate separate portions of the cushion component supporting the patient. In one embodiment, the air bladders can be coupled using plastic snaps, other firm yet flexible coupling devices, or other suitable elements as otherwise described herein.
An (optional) supporting frame, suitable for holding the cushion component on a seat or table, such as including a treated piece of wood, a firm plastic, or another firm material. In one embodiment, the frame can include a pommel disposed in a center front of the frame (and the air bladder layer can include a space into which the pommel can intrude), so as to provide a barrier against the patient accidentally sliding off of the cushion component and falling onto a floor.

In one embodiment, the cushion component can be coupled to (or can include) a set of electromechanical elements disposed to be controlled by the control system component, such as one or more of: a set of pumps and vents coupled to the air bladders, suitable for inflating/deflating the air bladders; a set of solenoids disposed to operate the pumps and/or open/close the vents in response to one or more control signals from the control system component; and otherwise as described herein. In such cases, the electromechanical elements can be disposed within the cushion component using rubber mounting, or other forms of mounting suitable to muffle sound and/or reduce an amount of noise experienced by the user when the pressure cushion system is operating.

### Control System Component

In one embodiment, the control system component includes
A pneumatic subsystem, coupled to the air bladders and suitable for inflating/deflating them, such as by pumping air into the air bladders (to inflate them) or by allowing air to vent from the air bladders (to deflate them). Each air bladder is coupled to a normally closed solenoid, which is pneumatically linked to an individual pressure transducer. Each air bladder is also coupled to an outlet for a quick disconnect. In one embodiment, the system can be quickly deflated using a sixth solenoid valve.
An electrical subsystem, coupled to the pneumatic subsystem and suitable for controlling and monitoring inflation/deflation of the air bladders. In one embodiment, the electrical subsystem includes a microcontroller, which can measure the pressure in an air bladder using that air bladder's individual pressure transducer. In one embodiment, the microcontroller operates under control of software disposed to perform the functions described herein.

### Pneumatic Subsystem

In one embodiment, the pneumatic subsystem can include one or more controlling solenoids for each air bladder, so as to couple a compressor/pump to that air bladder and a vent/exit from that air bladder. The compressor/pump and vent/exit associated with each air bladder can be suitable to inflate/deflate the air bladder under control of the electrical subsystem, such as by a microcontroller operating under suitable software control.

In one embodiment, the pneumatic subsystem can include a set of solenoids, each coupled to one (or more) of the air bladders and disposed to open/close an input (such as from a pump) or an output (such as to a vent/exit) coupled to its associated air bladder. In such cases, each such solenoid can be coupled to a pressure transducer disposed to measure pressure in the air bladders. The pressure transducers can include a set of piezoresistive strain gauges each disposed to be pneumatically coupled to an associated one (or more) air bladder; a differential voltage associated with each one (or more) of the strain gauges can provide information from which pressure in the associated air bladder can be determined.

In one embodiment, the pneumatic subsystem can be disposed to simulate zero pressure with respect to each one (or more) of the air bladders, so as to determine an offset voltage generated by its associated strain gauge. For example, to simulate zero pressure at a bladder, the pneumatic subsystem can be disposed to open a vent/exit from that bladder, or from multiple such bladders at once. The offset voltages might be associated with an ambient environment, imperfect fabrication, or other influences. In such cases, the electrical subsystem, or other controller element, can be disposed to record and maintain the offset voltages and compensate therefor during operation.

In one embodiment, airflow to the cushion component can be controlled using a normally closed solenoid associated with each one (or more) bladder and disposed to receive a voltage to open a value to inflate/deflate that bladder (or multiple such bladders). To open each such solenoid the electrical subsystem can be disposed to receive information from the pneumatic subsystem, so as to detect when the voltage has successfully opened the valve. For example, relevant information can include one or more of: a change in solenoid current in response to actuation; a measurement of current applied to a pump associated with the solenoid; a measurement of pressure at the bladder, such as pressure leading on the valve controlled by the solenoid; or otherwise as described herein. The electrical subsystem can determine an approximate minimum amount of power to apply to the solenoid for the valve, in response to information it receives from the pneumatic subsystem, and can adjust an amount of power it applies when attempting to open the valve.

In one embodiment, a vent/exit from the pneumatic system can be routed into a portion of the cushion component, such as a set of air passages or air cells in the foam element, so as to provide active cooling in areas where the patient is sitting. This can have the effect that the patient's sitting or lying on the cushion component does not unduly heat the cushion component, as active management of the air bladders can provide cooling to those areas being heated by the patient's natural body heat or pressure on the cushion component.

### Electrical Subsystem

In one embodiment, the electrical subsystem includes a relatively small, relatively low power processor having program and data memory and operating under control of software therein. For example, the program and data memory might be volatile or nonvolatile, combined or separate, internal or external, logically local or logically remote, or otherwise as described herein. When the program memory includes at least a portion that is volatile, the processor can be disposed to receive at least a portion of its program code from elsewhere upon startup. For example, the processor can be coupled to major electrical subsystems, and the program code it executes can be disposed to coordinate one or more electrical subsystems so as to provide efficient and/or safe control of pressures within the air bladders.

In one embodiment, the electrical subsystem can be disposed to receive information from the pneumatic subsystem, such as using electrical signals (such as voltages, other suitable electrical/electronic techniques, or as otherwise described herein). For example, the electrical subsystem can receive information with respect to pressure applied to regions of the cushion component, such as to individual bladders.

In one embodiment, the electrical subsystem can be disposed to measure pressure applied to each one (or more) of the air bladders in response to pressure measured with respect to the pumps and/or vents associated with each associated air bladder. The electrical subsystem can be disposed to obtain an accurate measure of external pressure, notwithstanding that the pressure measured with respect to the pumps and/or vents can be substantially inaccurate. In such cases, the electrical subsystem can include a processor operating under software control. The software can use an artificial intelligence or machine learning technique to determine parameters by which it can provide a substantially accurate measurement in response to relatively inaccurate measurements received from the pumps and/or vents.

In one embodiment, the electrical subsystem can also be disposed to measure a portion of the pressure measured at each associated air bladder in response to the user's sitting or lying thereon, versus a portion of the pressure applied to each associated air bladder in response to the backpressure from the bladder surface. In such cases, the electrical subsystem software can use a Kalman filter, or another suitable technique, applied to a set of individually unreliable information, to obtain a relatively reliable collective evaluation of what portion of the measured pressure is in response to each source.

In one embodiment, the electrical subsystem can also be disposed to apply electrical signals to elements of the pneumatic subsystem to control the latter. For example, the electrical subsystem can be disposed to inflate/deflate each one (or more) air bladders in response to one or more of: user movement; changes in an ambient environment, such as air pressure or humidity; changes in movement of the pressure cushion system, such as when used in a wheelchair being moved over a rough surface, or otherwise as described herein.

### Further Detailed Description

In one embodiment, a pressure cushion system (such as shown and described with respect to FIG. 1 at 001) includes a cushion component (such as shown and described with respect to FIG. 2) and a control system component (such as shown and described with respect to FIGS. 7-8).

### System Elements

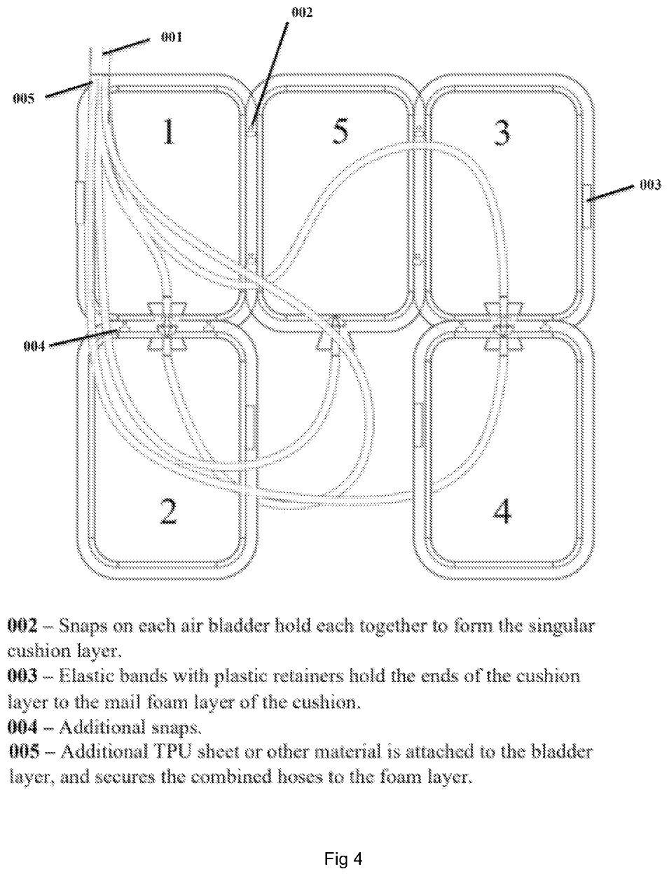

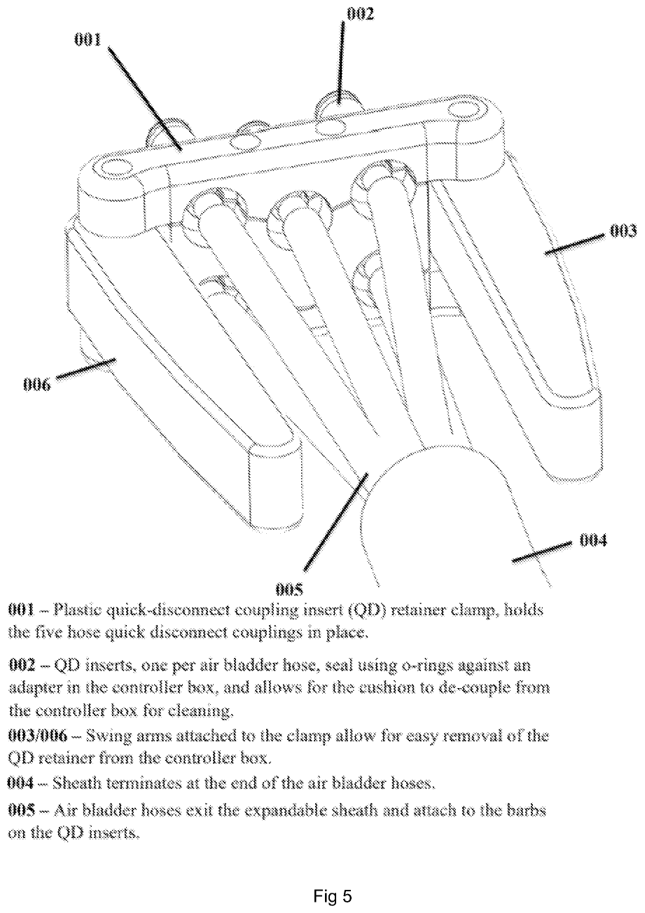

In one embodiment, the cushion component can include a sheet (FIG. 1, 001), typically made of a fabric or fabric like substance suitable to protect other elements of the cushion component from spills or other interference with the cushion component, a form of foam (FIG. 2, 004) and/or other padding material either configured in multiple layers of varied foams or consisting of one foam layer suitable for supporting the patient without direct contact with the air bladder layer, a layer of a configuration of a set of air bladders (FIG. 4) conjoined to form a cohesive unit, as well as an optional layer of some form of supporting frame (FIG. 2, 007) suitable for holding the cushion component on a seat or table typically made of a thin rigid water proof structure akin to a treated piece of wood, a firm plastic, and or any other material that conforms to the supporting frame described herein. For example, the air bladder layer can have five air bladders, each be disposed to support a separate region. The air bladders can be coupled to the control component using flexible tubing (FIGS. 4 and 5, 005), such as made of hose or tubing which can withstand relatively high amounts of air pressure. These tubes can couple to the air bladder layer to the control component using a quick disconnect interface.

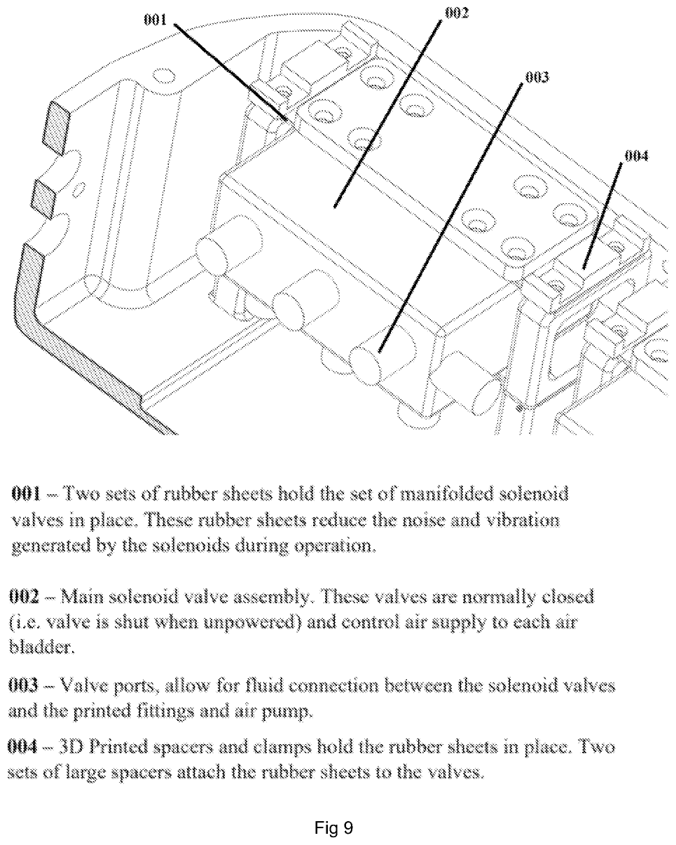

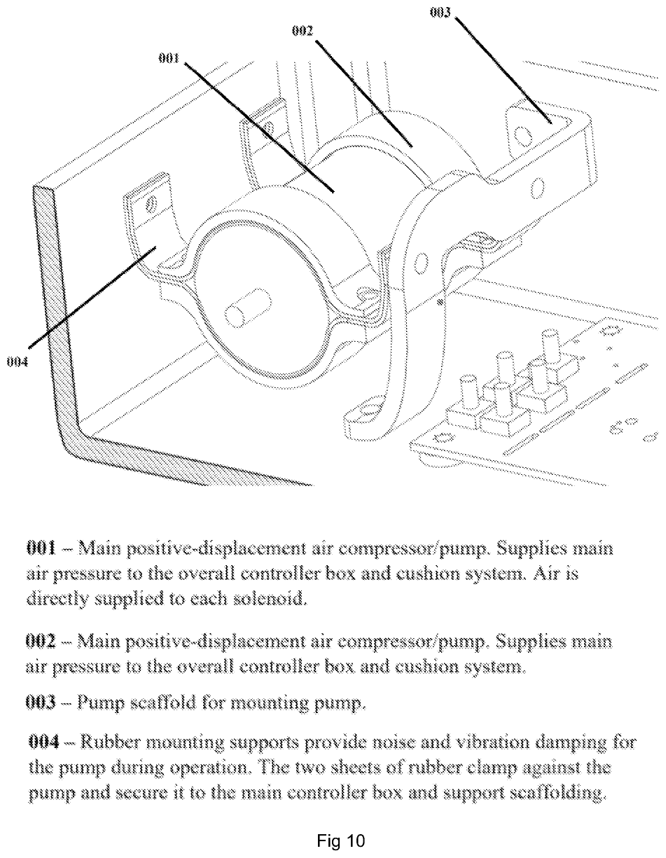

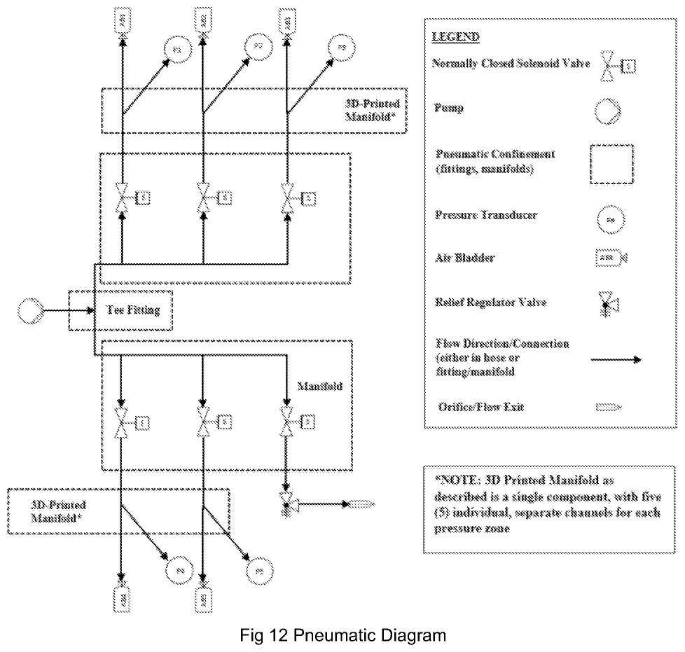

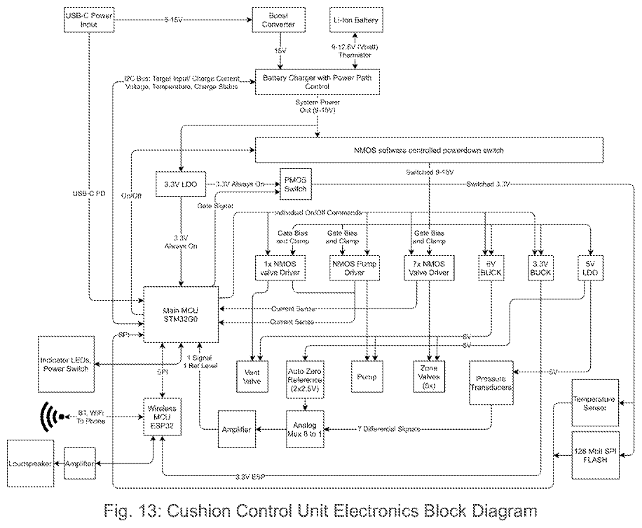

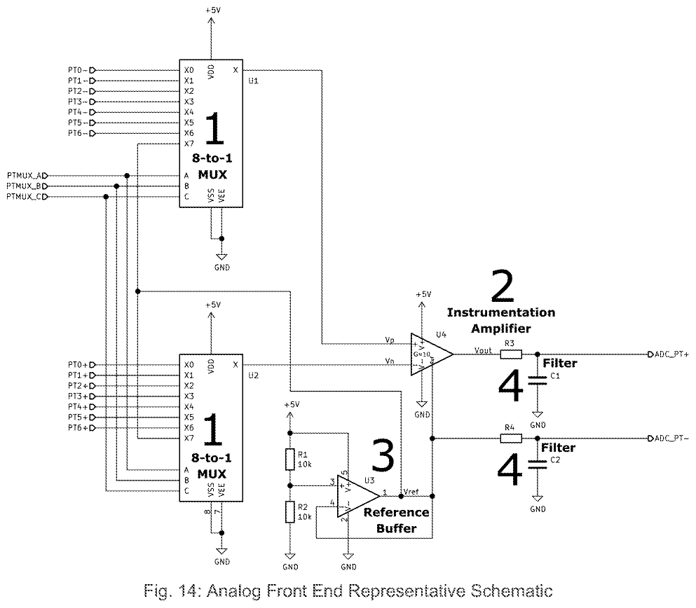

In one embodiment, the control component can include a pneumatic subsystem (FIG. 12) and an electrical subsystem (FIG. 13) (FIG. 14) disposed to control and monitor the inflation and deflation of the air bladders (FIG. 4) in the cushion component. The pneumatic system (FIG. 12) is composed of an air compressor (FIG. 10, 001) or pump (FIG. 10, 001) that supplies air to a set of manifolds (FIG. 8, 002), normally closed solenoids, one for each air bladder (FIG. 4), that control air flow in and out of the air bladders. Each solenoid is pneumatically linked to an individual pressure transducer (FIG. 12) and outlet from the box in the form of a quick disconnect (FIG. 7, 007). The cushion component attaches to the control box via the quick disconnect feature. For system deflation, a dedicated relief valve (FIG. 9, 003) is connected to a sixth solenoid valve. When opened in combination with a second valve, the air bladder in line with both opened valves will vent.

In one embodiment, the electrical system and main circuit (FIG. 8, 003) can control the pump and solenoids. Pressure transducers or monitors (FIG. 12) can log pressure data from each of the air bladders, and, using user presets or an automated machine learning algorithm can control the inflation and deflation of the air bladders, such as by operating the pump and solenoids in tandem. For example, the on-board control system can receive responses from the user using a cellular app to modify control behavior, and can be used to select various pressure setpoints and inflation sequences.

### Cushion Component

The cushion component is arranged for a user to sit upon or lean back upon (when positioned on a chair or other seating or resting surface) or to lie down upon (when positioned on a bed or other lying surface). For example, the cushion component can include (A) a fabric like sheet layer that works to provide form and house the various other aspects of the cushion component, (B) a bladder layer, arranged and coupled to devices so as to sense and respond to pressure from the weight or movement of the user, as described herein, and (C) a foam and or padding layer, arranged to provide a cushion between the user and the bladder layer. The cushion component can also include (D) a command element, arranged to receive input commands from the user, or a caregiver, so as to optimize the user's comfort and minimize any adverse medical conditions that might occur due to the user's relative immobility. The cushion component further can also include (E) a rigidizer or a form of thin rigid structure, typically a piece of wood, that acts as a support for the user to sit on.

### Bladder Layer

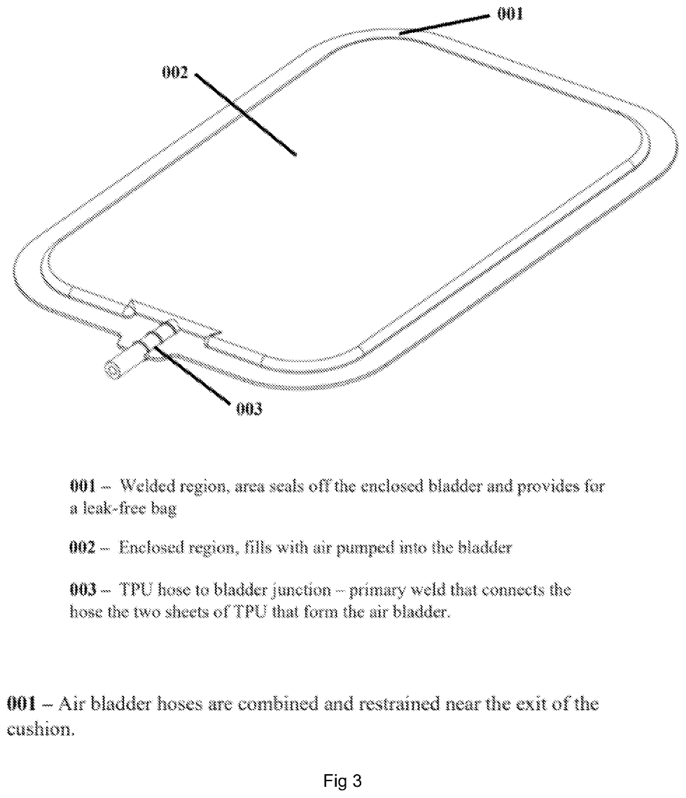

In one embodiment, the bladder layer (FIG. 4) of the cushion component can include multiple individual bladders (FIG. 3), in one configuration five bladders, but it is possible to enact the same or similar effects with a larger or smaller number of bladders, each preferably including thermoplastic polyurethane (TPU) sheets and preferably having a rectangular perimeter with rounded corners (FIG. 3). Each individual bladder can be coupled to a circular hose (FIG. 3, 003), preferably sealed by RF welding so as to prevent air leakage. For example, the TPU bladders can have the dimensions of 8 inches of length by 5.5 inches of width for the inflatable portion of the bladder, and 8.75 inches of length by 6 inches of width for the entire dimensions of the bladder, including the RF welded seam of the bladder. When inflated the bladder takes up the width dimensions of 7 inches in length by 3.875 inches in width and the entire RF welded device takes up the dimensions of 7 inches in length by 4.5 inches in width. Each bag is created by first taking two pieces of TPU sheet within and welding them together with a circular hose. The TPU sheets are then welded together a second time with one another to form the bladder-like pocket from the TPU sheets.

In one embodiment, the bladder layer (FIG. 4) includes five (or another number) TPU custom welded bladders. These bladders can be connected to one another by snaps in the order shown in (FIG. 4, 004). The snaps can be located directly on the overlapped welding of the bags (FIG. 4, 004). The snaps can be applied by piercing through the sheets and pressing them together. For example, bag 1 can be layered overtop of bags 2 and 5, bag 3 can be layered above bags 4 and 5. The upper layer of bags (1 and 3) can have the male end connectors, the bottom layer of bags can have the female connectors. Each bag can be connected to two other bags by two sets of snaps. Within the bladder layer there is also a supporting layer of TPU material that is located underneath bag 1, which is used to connect the tubing to the bladder layer itself. It is connected to bag 1 with 4 sets of snaps and is connected to both bag 2 and bag 5 by 1 set of snaps. The support layer has 3 zip tie mounts connected to the top of the support layer by an adhesive glue. “Zip ties” are wrapped around the hoses and through the mount to hold the hoses in place. There is a small offset between the bags that are connected top-to-top, so that the hoses don't overlap. The bladders can further be arranged in any number of configurations.

In one embodiment, the bladder layer is connected to the control component of the unit (FIG. 7, 001) (FIG. 2, 001). The control component is able to monitor various aspects of the cushion component as described herein. For example, the primary method for the data collection of the control component, and the primary input device for the control component can include the bladders. The bladders are connected to the control component via five (or another number) air hoses (FIG. 4), which can be directly connected from the air bladders to the control component via a quick disconnect device.

The air hoses can be welded to the bladders and all of them run through a slit in the back right of the cushion element through the sheet (FIG. 4, 003). The associated air tubes are run through a protective tubing sheath (FIG. 2, 001), which is typically made of a polyurethane sleeve with a 1-inch diameter. The quick disconnect (FIG. 5) feature can allow for the separation of the tubing aspect of the cushion component from the control component for cleaning and maintenance of components of the pressure cushion system. The connection between the control component and the cushion component can thus include being the interface between the quick disconnect aspect on the outside of the control component (FIG. 7, 001 & 007).

### Control Component

In one embodiment, the control component can be disposed to sense the position of the user, possibly over a period of time, and to control the stiffness of individual portions of the cushion component in response thereto, as described herein. For example, the control component can include a computing device, arranged and coupled to elements of the cushion component so as to (A) receive sensory data from the cushion component, and operating under control of software elements arranged to (B) process that sensory data, (C) determine one or more medical conditions associated with the user, (D) determine one or more treatments appropriate to those medical conditions, and (E) control one or more elements of the cushion component to apply those treatments.

### Pneumatic Mechanical Function

In one embodiment, the control component consists of a physical shell containing five (or another number) independently controlled pressurized zones corresponding to associated air bladders (FIG. 4). For example, each zone can include a normally closed solenoid valve for restricting flow within the zone (FIG. 8, 002), fluidly connected to a pressure transducer (FIG. 12) to record in-line pressure in the zone and an air bladder connected via flexible hose with an in-line quick-disconnect junction (FIG. 7, 007). A positive-displacement air compressor or pump can be connected to all of the pressure zones to provide air to each zone. The inflation and deflation of the air bladders can control the relative local thickness on the surface of the cushion component and can allow for the continued redistribution of the user's weight on the cushion as the bags inflate and deflate based on user preference, or as directed by the control component (such as using a machine learning algorithm, as described herein).

As further described herein, the control component can dispose individual bladders to support the user, in response to where the user sits, lies, or moves. The cushion can support portions of the user's body when the user sits or lies on the cushion, whether or not the user moves about or is stationary. When the user moves about, whether due to normal movement, due to fidgeting, or due to discomfort or pain, the cushion can detect the user's new position and adjust the amount of inflation/deflation of each bladder to support the user's new position. When there are multiple bladders, as described herein, the control component can adjust the amount of inflation/deflation of each bladder to support the user's body, particularly those hard portions of the user's body such as bony structures that might press upon the user's musculature and lead to pressure sores or other medical conditions.

As further described herein, the control component can adjust the amount of pressure provided by each individual bladder to provide a distinct amount of support to each portion of the user's body. Thus, when the user's weight, particularly those hard portions of the user's body such as bony structures, exerts pressure on the cushion, the pressure provided by each individual bladder can respond by exerting pressure to support the user's body. When the user moves, the pressure provided by each individual bladder can adjust to support the user, such as by dynamically balancing or rebalancing pressure underneath the user.

As further described herein, the control component can also adjust the amount of pressure provided by each individual bladder to massage the user's musculature to promote blood flow and to reduce seating fatigue. Thus, the control component can adjust the amount of pressure provided by each individual bladder to provide a difference in the amount of support to individual bladders, which can be felt by the user as a change in pressure at different places in their musculature. The difference in the amount of support can be a positive/negative pressure difference, which can be moved about to affect different portions of the user's musculature. The pressure difference can be cycled through a sequence of bladders periodically, can be moved about the set of bladders randomly or pseudo-randomly, or otherwise as described herein.

As further described herein, the control component can detect changes in amount of pressure applied to each bladder due to weight applied by the user. The control component can determine one or more patterns of activity associated with the user's movement, such as when the user gets up from or sits/lies back down upon the cushion and/or support, when the user deliberately moves to a new position or when the user is fidgeting, or when the user fails to move for a substantially period of time without having left their seating/resting area.

In one embodiment, the control component can (A) detect a sequence of weights applied by the user's body to one or more of the bladders, (B) determine a pattern of activity being performed by the user in response to that sequence of weights, and can (C) perform one or more actions in response to the detected pattern.
One such pattern might include when the total pressure applied by the user to the set of bladders rapidly decreases from the user's normal weight to nearly zero and stays that way for a period of time. In response thereto, the control component can conclude that the user has lifted themselves from the cushion and/or support. In such cases, the control component can adjust itself to an inactive/off state. The control component can perform the opposite function and adjust itself to an active/on state when the user sits/lies down thereon.
Another such pattern might include when the user's center of mass moves steadily toward an edge of the cushion and/or support, such as when the user might be sliding off a wheelchair or similar device. In response thereto, the control component can conclude that the user has lost stability and is about to fall off the edge of the cushion and/or support. The control component can adjust pressure on the bladders near the edge of the cushion and/or support, to provide a ledge or restraint preventing the user from falling off the edge of the cushion and/or support.
Another such pattern might include when the user's movement is very limited over a period of time, or when the user does not perform a medically recommended number of “offloads” (lifting themselves from the cushion and/or support and returning to a sitting position) in a selected period of time. In response thereto, the control component can conclude that the user does not have adequate mobility and will suffer one or more pressure sores or other untoward medical conditions without their musculature being massaged. The control component can adjust pressure on the bladders to provide an appropriate amount and location of massage on the user's musculature, to prevent the user from suffering pressure sores or other untoward medical conditions.

In one embodiment, the valves (FIG. 12) can be used to maintain pressure within the air bladders whether energized or de-energized. For example, when the valves are normally closed, if de-energized the bags maintain latent pressure. In such cases, to inflate a bladder, the control system energizes the pump (FIG. 10, 001) and the valve (FIG. 12) corresponding to the air bladder selected for inflation. This causes the normally closed valve to open, allowing for air to enter the valve and pass into the air bladder (FIG. 12) and pressure monitoring system. Pressure transducers (FIG. 12) on the main control board can monitor the common pressure within a single pressure zone; the control loop can shut the valve and de-energize the pump once the desired pressure in the air-bladder is reached.

Similar to inflation, the control loop can also manage bag deflation. This can be accomplished using a dedicated relief valve (FIG. 12) with an in-line regulator (FIG. 12). The use of a regulator allows for tuning bag deflation rate. A tighter regulation causes longer time of bladder air deflation as opposed to a more open state. This parameter can be used to tune the apparent rise and fall of the seat cushion to the user. However, the in-line regulator can be omitted in favor of a fixed orifice.

In one embodiment of the deflation apparatus (FIG. 12), the exit orifice of the pneumatic system (Pneumatic Diagram) can be routed back into the cushion to provide active cooling to the user as system air will vent directly into the cushion. With an additional sixth hose routed through the hose sheath as shown (in cushion diagram) venting can be provided into and around the foam layer of the cushion and reduce excessive heat and moisture buildup experienced by the user.

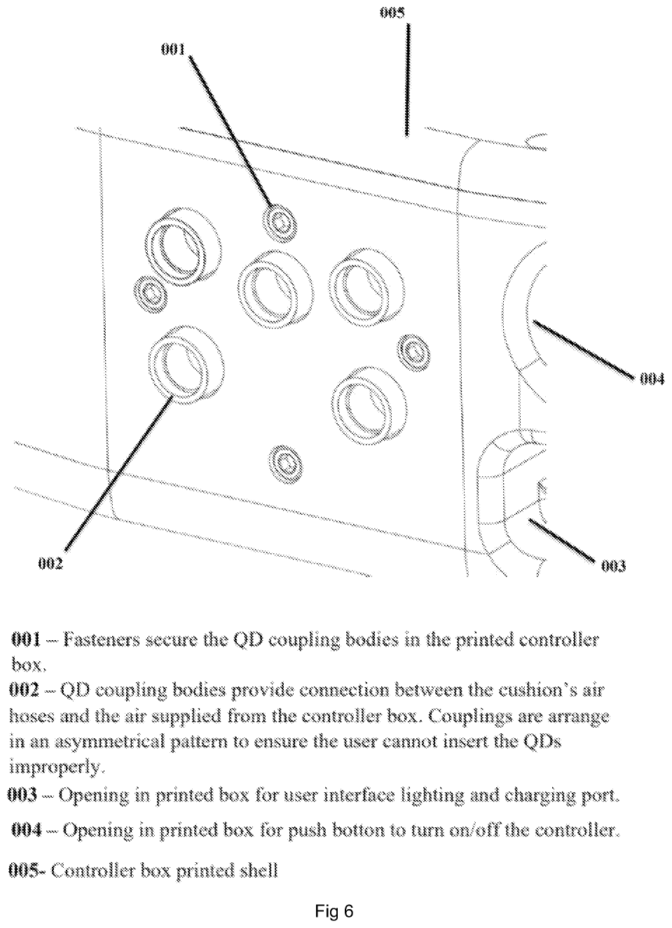

In one embodiment, the circuit board can receive data on the current state of pressure in the bladders using board mounted pressure transducers (FIG. 12) (FIG. 8). For example, there transducers can be fluidly connected to a pneumatic manifold (FIG. 12) which can be made out of a Stereolithography (SLA) 3D printed resin. In the pneumatic diagram this is represented by the upper and lower “manifold” dashed boxes. Note that this is a single printed manifold for the five (or another number) separate pressure zones printed within the part. The printed manifold (FIG. 12) can provide a common junction between the assigned pressure transducer (FIG. 12) on the circuit board, the outlet port on the solenoid block, and the quick-disconnect port for a given zone. The pneumatic manifold can be manufactured with 3 barb fittings per inflatable bladder (FIG. 4). The larger barbed fitting can be coupled to the output of the solenoids (FIG. 9). The two smaller barbed fittings can be fluidly connected to the larger barb and can be identical REF. One of the two smaller barbed fittings is fluidly connected to the transducer and the other is routed to the quick disconnect assembly (FIG. 6). The pneumatic snap on quick disconnects (FIG. 6) can be clamped into a PETG fused deposition modeling (FDM) 3D printed housing that can arrange them in an asymmetric pattern (FIG. 6), so as to adapt to the cushion component quick disconnect interface. This can help ensures that the user only installs the cushion into the control housing in the intended orientation.

### Sound Damping

In one embodiment, to mitigate sounds experienced by the user from electromechanical devices in the control component, both the positive displacement pump (FIG. 10) and normally closed pneumatic solenoid valves (FIG. 9) can be suspended on rubber housings (FIG. 10, 004) (FIG. 9, 001); this can have the effect that the hardware is not rigidly connected to the 3D Printed PETG control box enclosure. Rubber sheets can be used for this purpose, preferably of medium durometer (˜50A). The pump can also be encased in a foam sheath to further dampen noise produced from operation. This foam can be clamped within the main pump clamp assembly (FIG. 10) to secure the damping hardware in place around the pump.

### High-Level Software Operation

In one embodiment, the control element can include a primary microcontroller (and any co-processors) operating under control of software disposed to maintain the pressure within the bladders at intended levels. For example, a set of pressure transducers can be disposed to monitor the current pressure in each bladder; if the pressure of a bladder falls outside a selected range (such as exceeding a predefined or an automatically-tuned threshold), the control system can correct this with one or more possible procedures. A duration of each such procedures can be determined by a timer, by watching for pressures to reach a selected threshold, and/or by using the Prediction subsystem outlined in the Machine Learning subsection to halt the procedure at a time for reaching a desired operating pressure.

For example, the control system can open the airway from one or multiple bladders to the pump and apply power to the pump. This state can be maintained for a time, and then the pump may be shut off, and the airway to the bladder/bladders may be closed.

For example, the control system can open the airway from one or multiple bladders to the vent and apply power to the vent. This state can be maintained for a time, and then the vent may be shut off, and the airway to the bladder/bladders may be closed.

For example, the control system can open the airway between multiple bladders by energizing the solenoids attached to both. This will cause the pressures between the bladders to equalize. This state can be maintained for a time, and then the solenoids may be de-energized to cancel the procedure.

### Machine Learning

In one embodiment, pressure transducers responsible for measuring the pressure of the air bladders might fail to be located directly in the air bladders themselves, or even sufficiently close to those air bladders to provide substantially accurate measurements. For example, the bladders might be located relatively far away from the transducers, with thin, flow-restricting tubing connecting the two pneumatic regions. This can have the effect that when the bladders are inflated/deflated, the distance between where pressure is measured and where pressure is desired to be known, as well as movement of air through these tubes, can cause a substantially difference between measured pressure and actual pressure. Moreover, closing the pneumatic valves used to fill the region while the air is still moving will cause an effect, wherein the momentum of the air will temporarily cause a region of increased pressure downstream and reduced pressure upstream; this can have the effect that the measurement of pressure downstream can differ significantly from measurement of pressure upstream.

As pressure transducers located within the cushion subsystem are not located in the pneumatic area that the control subsystem is trying to control, using the reported pressures without adjustment by the control subsystem can result in incorrect pressure-adjustment behavior. If the control subsystem only causes pumping/venting of a bladder until the measured pressure reaches a selected target, the actual pressure can stabilize at a different value from (above or below) that target, instead of precisely at that target. If it were possible to precisely model behavior of air flow in the cushion component by precisely controlling geometry of air flow of the unit cushion component, it might be possible to use a fixed equation to negate this effect; however, any change in the geometry of the pneumatic pathways to or from the air bladders would likely cause any such fixed equation to be subject to substantially change.

In one embodiment, the control subsystem can use a predictor system to identify a relationship between measured pressure and actual pressure. For example, the predictor system can use an artificial intelligence or machine learning technique to approximate a set of fluid-dynamics equations describing a relationship between measured pressure and actual pressure, such as described herein. This can compensate for differences between pneumatic systems in distinct production units.

In one embodiment, the predictor system can determine a selected function of measurement inputs and a set of weights, so as to provide a predicted measurement. In such cases, the dynamic nature of the selected function can have the effect that particular units selected for the inputs and outputs should not make any substantially difference, so long as those units used do not change during operation, as the predictor system should be able to adapt to whatever units are used.

In one embodiment, input measurements for the predictor can include, but are not limited to, measured pressure at the time of the prediction request, measured pressure prior to the start of the current pump or vent operation, and how much time has elapsed between the start of the pump or vent operation and the time at which the prediction request is made. Other and further input measurements could be used in addition to or in lieu of these selected inputs.

In one embodiment, selected weights can include values selected in response to physical behavior of the unit. For example, weights can be selected in response to iterative backpropagation of measurements, such as described herein. In one embodiment, two separate sets of weights are maintained for each air bladder, one for inflation and one for deflation.

As described herein, an output from the predictor represents the predicted pressure that the bladder will settle to after the current pump or vent operation is terminated. While the predictor is primarily described with respect to pounds per square inch (PSI) measurements provided by the pressure transducers, there is no particular requirement for any such limitation. Any consistent units may be used.

In one embodiment, the particular backpropagation technique for determining the weights can be similar to that described by the following pseudocode. This procedure can be performed substantially every time a pump or vent operation is terminated.

```
  input_measurements = current_system_state( );
  input_prediction = Prediction(input_measurements);
  sleep(300ms)
  final_measurement = current_zone_pressure( );
  perr = input_prediction − final_measurement;
  corr = perr * 0.004;
  if (corr < −0.1)
    corr = −0.1;
  if (corr > 0.1)
    corr = 0.1;
  for (weight in current_weights) {
    // Get the partial derivative of the prediction with respect to this
single weight
    pderiv = partial_derivative(weight);
    new_weight = weight + pderiv * corr;
    }
```

As described herein, this procedure can have the effect of backpropagating the final prediction error back to a correction on each weight, where the correction is proportional to the effect each associated weight had in the final prediction. This can enable a relatively fast convergence rate, requiring only about several hundred cycles to stabilize to reasonable predictions. While this predictor is primarily described with respect to backpropagation, other and further training algorithms may be used in addition or in lieu thereof when they are able to converge to a stable prediction.

In one embodiment, the input measurement values used for prediction start with: the pressure of the bladder at the start of the pump or vent operation (denoted herein as A), the pressure of the bladder at the time of the proposed pump or vent halt (denoted herein as B), and the inverse of the time spent in the operation in seconds (denoted herein as C). For example, a prediction function can include a quadratic function of these parameters, thus:

f(a,b,c,w₀,w₁, . . . w₉)=w₀+aw₁+bw₂+cw₃+aaw₄+bbw₅+ccw₆+abw₇+acw₈+bcw₉
where f( . . . ) is the predicted value.

For example, the prediction function f( . . . ) is thus responsive to changes in the variable a (in the terms aw₁, aaw₄, abw₇, and acw₈), and similarly to changes in the variables b and/or c (in other terms). While the prediction function f( . . . ) is primarily described herein with respect to this one quadratic function, there is no particular requirement for any such limitation. For example, a different quadratic function, a cubic or quartic or another polynomial function, or a function having other or further types of terms or computation could be used. The inventors have found that the particular function f( . . . ) has a preferred degree of prediction power and the number of required samples involved for convergence.

As described herein, in this particular implementation (others may be used), the variable b represents the pressure (provided to the control system from the transducer) that loosely corresponds to the pressure in the bladder at the time. This value can be generally imperfect, as the transducer might be physically located at a substantially different point in the pneumatic system than the pressure the system generally prefers to be measuring. The system generally prefers to be measuring the actual value of the pressure inside the bladder; therefore, it would be advantageous to have a method of refining the value given by the variable b into the actual value of the pressure. The output of f( . . . ) represents this prediction (in the same units as the variable b), and can be used frequently during the operation of the device.

### System State Detection

In one embodiment, the weights that this process generates can also be used to monitor the overall performance or health of a pressure cushion system. If parameters of the electrical or mechanical components of the pressure cushion system change over a time duration, this can have the effect of inducing a change in the weights over that time duration. If the weights are observed to change too much (such as, more than a selected threshold) over that time duration, the prediction subsystem can determine that the pressure cushion system has physically degraded too much; the user, one or more caretakers, or the manufacturer, can be alerted.

In one embodiment, when the pressure applied to the transducers is determine to be too low (such as, lower than a selected threshold), or the pump is determined to not affect the pressure when given a chance to for a small period of time, the prediction subsystem can determine that the system is either defective, or can determine that the box assembly has become separated from the cushion assembly. In either such case, the user or one or more caretakers can be alerted.

### User State Prediction

### Estimating External Pressure

One class of information that we can get from the control system is regarding the external force applied to the bags of the cushion. With this information many sub-features can be realized. This will first describe some different ways this information could be collected, and afterward we will describe the different ways for the information to be used.

In one embodiment, the system can infer external pressure (force on a user) from a measurement of volume and a measurement of internal pressure (pneumatic pressure). The system can use a valve (such as a solenoid valve) to allow air within a selected volume under test to equalize with another air body (such as a selected air volume) that has a known volume. The system can measure an equilibrium pressure reached by equalizing air pressures. In response to the internal pressure, the volume, and the equilibrium pressure, the system can determine the external pressure.

In one embodiment, the system can keep track of an amount of an expected air mass moved over time. In response to the expected air mass moved and the internal pressure, the system can estimate the external pressure. For example, the system can track the contained air mass by one or more data merging approaches disposed to find agreement in multiple data points received from individual sources that are believed to be unreliable (such as one or more sensors). These data merging approaches may be either statically defined, or they may be dynamic and able to adjust to different observed system behavior. These data merging approaches may also employ a simulated version of the pneumatic system to aid in finding agreement between the unreliable data-points.

### Merging Many Uncertain Information Inputs

Without a direct way to measure the external force applied to the bladders in the cushion, it is possible to build a guess for this value (on a per-bladder basis) which can be refined over time as various sensor readings are collected and combined.

One numerical method that may be used to merge these input hints into a coherent guess is known as a Kalman Filter (alternatively, an Extended Kalman Filter, or one or more other techniques for combining multiple measurements that are only partially certain or trusted into a single trusted measurement) may be used to provide a live prediction of the state of the system in response to one or more such input hints. This state may include pressures, air flows, temperatures, forces, masses, and/or other metrics. Values associated with this state do not necessarily need to be able to be directly measured. For example, this state may be initialized to an expected initial state and allowed to correct itself over time in response to sensor data. The Kalman filter can establish, for each potential input data stream (such as one or more such data streams described herein), a predicted value for that data stream. The Kalman filter can also establish, for each potential input data stream, a relationship between any error seen in the sensor data (thus, one or more differences between perceived and predicted values) and how that error should be propagated to update the predicted value.

While this Application primarily describes merging multiple uncertain information inputs using a Kalman Filter, there is no particular requirement for any such limitation. In alternative embodiments, other and further numerical processes may be used in additional to or in lieu of a Kalman Filter. For example, one such alternative can include Bayesian updates.

### Some Selected Uncertain Information Inputs

Selected uncertain information inputs can include one or more of the following observations. When the predicted value is expected to have a known value (or a value in a known range), the predicted value may be set to this value or range, or alternatively, moved closer thereto; when the predicted value is expected to have a known derivative (or a derivative in a known range) the predicted value may be shifted to match the derivative having the expected value or in the expected range.
Differential pressure sensors and/or flow meters can be used to measure the flow of air through the hoses to the bladders. The current drawn by the pump can be used to infer the volume of air it is delivering at the pressure that is measured by the transducers. Predictions can be made using the pressure/time curve read by the primary pressure transducers while a bag is filled or emptied. This can be compared to a calibrated pressure/time curve for the unit, and any differences will indicate applied external pressure.
Force gauges can be applied to the bladders to measure how much they have stretched, and this value can be compared to the internal pressure measure to estimate the external pressure exerted upon the bladders. (The phrase “internal pressure” generally refers to a measure of pneumatic pressure applied to a bladder, such as a pressure exerted by gas filling the bladder. The phrase “external pressure” generally refers to a measure of force applied by the bladder to the user, such as an upward force counteracting the user's weight. As otherwise described herein, the force applied by the bladder to the user is responsive to the pressure exerted by gas filling the bladder and can be modulated by a foam cushion element disposed between the bladder and the user's body.) A pressure mapping device could be employed on top of, or beneath the bladders to directly measure the externally applied pressure. Additional air bladder(s) that are not pressure-regulated may be used as sensing elements in order to detect externally applied pressure. The strain of the bladder may be measured by applying acoustic energy to the pneumatic lines leading to the bladder. Variations in the frequency response of the system may indicate variations in the strain of the bladder.
The pump and/or the vent may be briefly activated for a controlled period of time, and the corresponding pressure transducer can be used to watch pressure response to this event. Variations in the external pressure of the bag will result in different pressure responses over time. One of the bladders may be deflated and inflated independently of the rest of the bladders. The speed of inflation may be monitored, and a load on the bag may be detected by a change in the inflation rate once the bag volume is increased enough for it to provide partial support to the load.

### Some Selected Known Scenarios

One or more known scenarios the pressure cushion system might encounter (for example, when the user sits down on the cushion subsystem, this can cause all or most of the bladder pressures to increase rapidly). One or more known scenarios can also provide hints as to the current state of the external pressure of the cushion. While this Application describes some selected known scenarios, there is no particular requirement for any such limitation; other and further such scenarios, and/or combinations thereof, are also possible. Moreover, information from more than one such scenario can be combined when so identified.
When all or a large number of bladders experience a rapid decrease in internal pressure, the external pressure can be predicted to be zero, or close to it.
When a rapid increase of pressure is observed from the bladders, the difference in pressure from a time to, before the increase in pressure, to a time ti, after the increase in pressure, can be predicted to be an amount of external pressure placed onto the bladders. This information can be interpreted as the weight of a load, such as either a user or some other weight, applied to a top of the cushion.
When a subset of the bladders of the device have had pressure rapidly removed, and other bladders have had pressure approximately equally rapidly applied, the external pressure can be predicted to have been removed from some bladders and applied to other bladders in a zero sum fashion, as the user has probably shifted their weight among the bladders. The control component may estimate the time derivative of externally applied pressure by measuring the derivative of the sensed pressure in bladders that have not been recently inflated/deflated.
When the pressure in a bladder is subject to variance over time, such as might indicate fidgeting or movement by the user, the cushion can be disposed to detect the presence of the user and thus to remain active so long as the user is present. For example, the sensors associated with the cushion can be disposed to determine a measure of how much variance in pressure occurs over a selected period of time and compare that measure of variance with a threshold. When the user is present on the cushion, the system can expect to see increased noise with respect to observed pressure data; when the user is not present, the system can expect to see nearly static pressure measured in the cushion. When the variance exceeds the threshold, the cushion can determine that the user is present, while when the variance is less than the threshold the cushion can determine that the user is absent. The measure of variance can be a standard deviation or other measure of how much change in pressure occurs over a selected period of time.
When the pressure in a bladder is modified, the corresponding shift of external force due to the user's shifting weight may be predicted, and counteracting pressure changes in adjacent bladders may be attributed to this action, and not to outside events.
When the cushion detects one or more changes in pressure for selected bladders that indicates the user has moved, the cushion can be disposed to determine whether the user has adjusted their posture with respect to the cushion. For example, when the user is sitting in a chair and slumps with their hips forward of their chest, this can exert undesired force on the user's spine. In such cases, the cushion can be disposed to use the bladders to exert an opposing force on the user's hips to push them back to a better posture. Alternatively, in such cases, the cushion can be disposed to indicate an alert to inform the user, medical personnel, or a caregiver, that the user is improperly positioned or otherwise disposed in a way that might promote an injury or other negative medical outcome.
   Use of Pressure and/or Volume Information

The cushion (or a processor coupled thereto) can be disposed to receive pressure and/or volume information and to determine one or more useful responses thereto.
The cushion can be disposed to model an external pressure, such as a force directed upward toward a user disposed on top of or leaning on the cushion, in response to an internal pressure and/or a movement or position of the user. For example, the cushion can use a Kalman filter or an Extended Kalman filter to determine a measure of the external pressure for one or more bladders in response to their associated internal pressure (such as at their pump/vent locations). For another example, the cushion can use a Kalman filter or an Extended Kalman filter to determine a model of the external pressure (such as a model using polynomial or exponential functions) in response to the internal pressure. In such cases, the cushion can determine the external pressure in response to the internal pressure and can alter the internal pressure to achieve a desired external pressure.
The cushion can be disposed to model movement (such as fidgeting or deliberate movement), position (such as body angle or posture), energy (such as a number of lifting or “offloading” exercises performed by the user in a selected period of time), or other information about external pressure, about the user, or about positioning and/or use of the cushion.

While this Application primarily describes determining a measure of external pressure (force on the user) in response to the internal pressure (pneumatic pressure on the bladder) using a Kalman Filter or Extended Kalman Filter, there is no particular requirement for any such limitation. In alternative embodiments, other and further numerical processes may be used in additional to or in lieu of a Kalman Filter or Extended Kalman Filter. For example, one such alternative can include Bayesian updates, artificial neural networks or other artificial intelligence techniques, or other techniques described herein.

The cushion can also be disposed to determine information about the user in response to pressure and/or volume information. For example, the cushion can be disposed to:
Determine if any or which one of a selected set of users is present on the cushion. When a first user exits the cushion, such as by getting off the cushion, and a second user sits/lies down on the cushion, the cushion can identify the second user, such as in response to their particular shape, posture, weight, movement pattern, or otherwise as described herein.
Determine the user's shape, posture, weight, movement pattern, or otherwise as described herein. Similarly, the cushion can be disposed to determine whether the user has changed their posture, gained/lost weight since a past use of the cushion, shifted their posture while using the cushion, or otherwise as described herein.
Determine whether the foam (or another padding disposed between the bladders and the user) has changed in compression, become frayed or otherwise degraded, been supplemented with another padding, or otherwise as described herein. For example, if a foam padding has become frayed or otherwise degraded, the cushion can be disposed to indicate an alert to the user, to medical personnel, to one or more caregivers, or otherwise as described herein. For another example, if the user has been positioned on the same location of the foam for too long, they can exert pressure to squeeze the foam to the point when they are sitting directly on the bladders without any substantially cushioning.
Determine whether the user has disposed themselves on the cushion in a different or an unusual position, such as whether the user is disposed on top of an anomalous object (such a wallet, keys, or a similar object), whether the user is fidgeting or moving an adequate amount, whether the user is lifting or “offloading” themselves an adequate amount, or otherwise as described herein.
Determine whether the user is disposed on the cushion at an angle, such as whether the user is disposed on a wheelchair and is tilting the wheelchair to offload therefrom. In such cases, the cushion can alter its force on one or more selected bladders to assist the user in performing the offload while reducing the likelihood of injury or other mishap.
Determine whether the user has shifted their weight forward. For example, this could indicate a change in user posture that places untoward pressure on sacral elements or other portions of the spine. Alternatively, this could indicate a change in user posture that presages a slide or fall, or a sliding fall, from the cushion.
Determine whether the user has any pelvic obliquities or other untoward or unusual positioning. In such cases, the cushion can assist the user with setup (whether performed initially or reperformed upon newly sitting/laying on the cushion).
Determine the user's position/movement at times associated with the user identifying pain or other medical conditions, or at times when the user was found to have developed a pressure sore, injury, or other medical condition. In such cases, this information can be used to train/retrain or improve one or more pressure responses by the cushion. The pressure responses can be used to relieve pressure on the user and/or to urge the user to position/reposition themselves to relieve pressure or otherwise to avoid pressure sores, injury, or other untoward conditions.
Determine locations where a user is sitting, what the user's posture is, one or more locations where the user’ center of gravity is (as each might change over time), on or within a known seating area, or otherwise as described herein.

The cushion can also be disposed to determine information about the bladders or foam, and/or how the user is sitting/laying thereon, in response to pressure and/or volume information. For example, the cushion can be disposed to determine and present (such as in a visual “heatmap”) a set of pressure information at selected locations on the cushion. This can be used to show the user, medical personnel, and/or one or more caregivers, how the user is sitting/laying on the cushion and whether the user's disposition on the cushion is more or less likely to lead to pressure sores, injury, or other untoward conditions.

Through the use of state estimation, a flow meter, or an accumulator with known volume and a known orifice diameter which fills whenever a bladder is adjusted and allows estimation of flow rate and bag volume, along with various other techniques of measuring volume, the surface pressure between the user and the top of the cushion may be estimated, as otherwise described herein. With this data there are numerous ways that we may provide value to the consumer including but not limited to:
Identifying different users who are sitting on a cushion through weight or positional variations as well as whether the typical user of the cushion has experienced any weight or postural fluctuations.
Since foam breakdown is known as a common problem for cushions, the cushion may use the trend over time of the bladders needing to inflate to a larger height and pressure in order to provide ample support for the user. This data may also be analyzed along with collected data such as total sitting time to provide more accurate estimates of foam health.
A problem which can lead to pressure injuries and back pain is the presence of anomalous objects, such as a wallet or phone, between a person and the cushion; any one or more of these can create a pressure spike. If the volume required of a specific bladder were to experience a significant shift in a short period of time, the cushion could notify the user to check for any objects.
Since people perform different types of offloads such as tilting a chair backwards, leaning, and lifting their body up with the arms of their chair, it is difficult for the cushion to determine how to react in these scenarios and detect the presence and quality of said offloads. If the volume in each bladder were known, the accuracy of offload detection would be improved and allow more tailored responses in the scenarios. One other benefit of this detection is that if someone leans to reach for something such as working in a garden, the cushion could detect this and not try and offload the pressure from the front of the cushion.
In situations where the center of gravity of the individual is estimated and monitored through the surface pressure estimation, an individual could be monitored for shifting their pelvis in a way that causes excess pressure on the sacral region known as “sacral sitting” or is sliding out of their chair in a manner that could indicate a slide fall is likely, the cushion could send notifications and also adapt its molding to try and reduce the effects of sacral sitting and slow down the slide fall.
When doing an initial setup with the custom molding, if a pressure map is not available the cushion may be able to detect seating issues such as pelvic obliquities where misalignment causes an uneven distribution of pressure. Through better estimation of seating habits, the algorithm that determines how the cushion reacts to improve pressure relieving and redistribution would have additional data which could better enable it to prevent pain from developing or pressure injuries from sitting. The scenario where foam is fully compressed by the individual sitting is known as “bottoming out”. This scenario causes very high-pressure spikes and when present is on the ischial tuberosities. If this has happened and an individual is left to be supported only by the bladders, it may reduce the effectiveness of the cushion. By knowing if this has occurred, a notification could be sent, and the cushion could be adjusted either automatically or by the user to accommodate. When viewing the app, a user may be informed about the current state and height of bladders which would help visualization of how their settings affect the shape and molding of the cushion. This would help visualize and ensure that the cushion is performing as expected.

### Adjustable Bladder Support to Provide Custom Molding

Through the adjustment of air bladders beneath the foam, the shape and properties of the foam which support a user is able to be varied in a way that allows the foam to react to a user's body as though it were custom molded. When the bladders compress the foam from the bottom, they increase the upward pressure, which can effectively lift the foam and increase the force that the user feels from the foam in that region. This method of custom molding enables adjustment to create different user profiles which enable the cushion to change how it supports the user in different scenarios. This method of custom molding also enables dynamic adjustment, so as to change the molding in response to user activity or other circumstances relating to the user's medical conditions, comfort, likelihood of slippage or falling, or other events.
The bladder support to provide custom molding can be adjusted over time as someone's body changes such as gaining or losing weight, tissue changes, or the many other ways that the ideal seating position might vary. The bladder support can be tuned in real time by a therapist when fitting the cushion initially, eliminating the need for long lead and expensive modifications from machined and other manufactured customization methods (which can involve equipment to make adjustments).
The bladder support to provide custom molding can be enhanced by surface pressure estimation to allow custom molding to change in response to postural issues (such as unbalanced pressure due to pelvic obliquities or amputations), as well as in response to stability issues (such as derived from reduced mobility that could occur from strokes or spinal cord injuries).
The bladder support to provide custom molding can be used to change custom molding in response to conditions that might progress rapidly (such as ALS), as the user might need different mobility devices or as the user's postural stability degrades (such as to where an ideal seating surface changes).
The bladder support to provide custom molding can also be responsive to other seating factors, such as the “dump angle” of the seat pan. Therapists or other medical personnel might desire, when setting the dump angle, to vary that angle (since different user activities might benefit from different dump angles). The bladder support can provide change to the pitch of the cushion, possibly allowing at least some of the issues related to dump angle to be mitigated.
   Information from Externally Applied Pressure

Other and further known scenarios involve the pressure cushion system having an interaction between an observed externally applied pressure (such as from a weight on the pressure cushion system), with an internally applied pressure (such as from an internally-generated pressure by the air bladders within the cushion component). While this Application describes some selected known scenarios, there is no particular requirement for any such limitation; other and further such scenarios, and/or combinations thereof, are also possible. Moreover, information from more than one such scenario can be combined when so identified.
When the externally applied pressure is reduced to zero, or nearly so, this information can be used to pause or shut down the pressure cushion system, either immediately or after a brief period of time, as the user can be inferred to have likely left the cushion component.
When the externally applied pressure is rapidly increased from zero, this information can be used to cause the pressure cushion system to power on, as the user has most likely sat down or lay down on the cushion component.
When externally applied pressure is determined to shift more frequently than average, this information can be used to predict that the user is uncomfortable, and the bladders in the cushion could be adjusted to increase comfort. For example, the cushion can measure applied pressure and determine a frequency of how many times pressure changes by more than a selected threshold amount; alternatively, the cushion can determine a standard deviation of how many times pressure changes by more than the selected threshold amount. The cushion can determine that the user is uncomfortable in response to the standard deviation being more than a selected threshold amount.
When externally applied pressure is determined to shift more than average, this information can be used to determine whether the user is present or not. For example, the cushion can measure applied pressure and determine a standard deviation of an amount of that pressure. When the standard deviation of the amount of pressure is more than a selected threshold, the cushion can determine that the user is present; when the standard deviation is less than the threshold, the cushion can determine that the user is absent.
When externally applied pressure is determined to be shifting very infrequently, or at least more infrequently than average, this information can be used to predict that the user is relatively comfortable, and the current bladder pressures can be saved for future use.
When externally applied pressure does not leave a given range of pressure for more than a selected threshold amount of time, this information can be used to predict that the user is substantially immobile. The pressure cushion system may alert the user or one or more caretakers.
When externally applied pressure on the bladders of the unit in one extremity (forward, rear, left, or right) is sufficiently greater than the mean pressure of all bladders in the unit, this information can be used to predict that the user is likely sliding off, or about to slide off, the cushion component. Alternatively, this information can be used to predict that the user is in a state of strong pressure displacement. The pressure cushion system can alert the user or one or more caretakers. In addition or instead, the pressure cushion system can modify pressures in the different bladders so as to shift the user back into a safer position.

A value of the mean externally applied pressure can be measured, and this information can be used to predict the weight of the user. The pressure cushion system can communicate the user's predicted weight to the user and/or one or more caretakers. When tracked over a time duration, the pressure cushion system can be disposed to provide a calibrated time-graph of the user's weight.
When the externally applied pressure is changing, the pressure cushion system can pause its normal pressure-control behavior, so as to prevent unnecessary wear on the pressure cushion system and prevent unnecessary battery or power usage.
When, for any particular bladder, the externally applied pressure is determined to never (or quite rarely) decrease below a certain threshold over the course of a long period of time, the information can be used to predict that the user might be exerting excessive pressure on a designated portion of the body. The pressure cushion system can be disposed to alert the user or one or more caretakers, as this might enable pressure sores to develop.
The pressure a user experiences when sitting on the device, can be inferred in response to the external pressure reading from a bladder, as well as the preexisting pressure dampening aspect of the foam configuration. This allows the pressure cushion system to systematically offset areas of high pressure for the user. Moreover, information derived from variations in the externally applied pressure can be used to predict when the user is sleeping or awake. The pressure cushion system can communicate the information to the user or one or more caretakers, such as using a graphical user interface.

There are a number of alternative uses for the cushion and/or support. For example, the cushion and/or support can be disposed for use by medical patients who are relatively immobile, either because their musculature is weakened, because they are restrained by medical devices, or because medical personnel would like them not to move selected parts of their bodies (whether because those selected parts are healing, because movement would disrupt or interfere with medical devices or with user healing, or because movement would cause undue pain to the user). For another example, the cushion and/or support can be disposed for use by persons who are confined for relatively long periods of time, such as drivers engaged in automobile travel, pilots or other personnel (such as medical or military personnel) who are engaged in activity that demands their full attention and for which they cannot move about. For another example, the cushion and/or support can be disposed for use by persons who are required by their activity to position themselves in an awkward or strained manner.

Users may read any of the below measurements via the user's mobile control unit. Users may also set desired thresholds for this information and be alerted using one of the methods described below if any measurements exceed the provided thresholds. The position of the unit can be estimated with a variety of methods, including GPS, Cellular positioning, adjacency to various wifi devices, and adjacency to various Bluetooth devices. The temperature of the user, the atmosphere, and/or moisture within the cushion component of the unit may all be measured directly with various sensors.

### Control Devices

The pressure cushion system can be disposed so as to allow the user, and/or one or more caretakers, to use an external device as a control input, or as a notification source or information readout. Such external devices can include, but are not limited to, Mobile Phones (Android™-based, iPhone™-based, or others), Tablets/Phablets, Laptops, Desktops, simple remote controllers (IR based, RF based, or others), audio-only interactive devices (Amazon Alexa™, Google Home™, or others). These devices can provide one or more user interface points and can allow the user and/or one or more caretakers a degree of control/output with respect to the pressure cushion system. The pressure cushion system can be disposed to allow one or more such devices to be connected using a wired connection, Bluetooth™, Wi-Fi, cellular, other forms of RF control, sonically, or by IR tranceiving solutions.

### Alternative Embodiments

Although this Application primarily describes one set of apparatuses, methods, and preferred techniques, in the context of the invention, there is no particular requirement for any such limitation. Other apparatuses, methods, and techniques, and related matters, would also be workable, and are within the scope and spirit of this description. After reading this Application, those skilled in the art would be able to incorporate such other techniques with the techniques shown herein.

This Application describes a preferred embodiment with preferred process steps and, where applicable, preferred data structures. After reading this Application, those skilled in the art would recognize that, where any calculation or computation is appropriate, embodiments of the description can be implemented using general purpose computing devices or switching processors, special purpose computing devices or switching processors, other circuits adapted to particular process steps and data structures described herein, or combinations or conjunctions thereof, and that implementation of the process steps and data structures described herein would not require undue experimentation or further invention.

The claims are incorporated by reference as if fully set forth herein.

### TECHNICAL APPENDIX

### Cover

The cushion component can be wrapped in a number of cover interfaces in order to contain the various other aspects found within this portion of the device. The cover aspect (FIG. 1, 001) can be a seating interface which includes a cover which works to maximize stretchability and breathability by using a thin lycra layer. This lycra layer is sewn to a thicker knit polyester fabric layer that covers the outer perimeter of the lycra layer while less flexible than the lycra, is still very porous and breathable. In this example of the cover aspect, the bottom of the cover (FIG. 1, 001) is composed of perforated non-slip expanded pvc fabric with a knit backing and is sewn to the knit polyester fabric. In order to properly affix the cushion onto a seating or laying surface, hook and loop straps are sewn into the bottom layer of the cover to secure it in place. The lower back right of the cushion cover is left unsewn between the pvc and polyester fabric to allow the hoses to pass through. An additional piece of fabric is sewn onto the outside of the knit polyester fabric to indicate the orientation of the cushion.

The cover can further be designed to consist of a knit polyester fabric with a rubberized non-porous material backing on the top and side layer. The rubberized knit polyester fabric is sewn to a perforated non-slip expanded pvc fabric bottom layer. The lower back right of the cushion cover is left unsewn between the pvc and polyester fabric to allow the hoses to pass through. An additional piece of fabric is sewn onto the outside of the knit polyester fabric to indicate the orientation of the cushion.

Typically the cover there will be an allowance for space if the user needs to insert a form of rigidizer; which can range from a piece of wood, to a plastic layer, to any other rigid and thin structure as shown in (FIG. 2, 007). This object, in one embodiment, would be placed inside the cover aspect (FIG. 1, 001), underneath the bladder layer (FIG. 2, 006) to provide some form of additional structural stability to the cushion component of the unit. The rigidizer can simply be added directly beneath the cushion component without needing to be added inside the cover aspect. The rigidizer can range anywhere within the dimensions of the cushion component. In most aspects it's dimensions will work to align with being insertable into the cushion component, however it is possible that the rigidizer may be slightly larger than the cushion component in order to provide additional stability to wider seating and or laying surfaces.

In addition to the various forms of cushion component covers, if the user requires it, a separate waterproof cover may be placed over the entire cushion component and or unit assembly. Such waterproof covers may be made out of polyester terry fabric, vinyl, polyurethane, silicon spray, or any other various waterproofing material layer.

### Foam

A foam layer is the top layer of the inner aspects of the cushion component (FIG. 2, 004). The foam layer can typically range anywhere from 2 to 4 inches in height and within the range of 16 to 18 inches in width and 16 and a half to 20 and a half inches in length. The foam layer (FIG. 2, 004) typically rests upon the bladder layer (FIG. 2, 006). The foam layer can consist of a range of various porous structures typically consisting of polyurethane. The foam layer can further be a layered structure and multiple other materials. The foam layer can range in dimensions from any combination of 16-24 inches in length, 1 to 4 inches in height and 16 to 20 inches in width. A foam layer provides the main immersive function of the cushion embodiment, as well as a mounting structure for the inflatable bladder layer. For heavier or smaller patients, the foam will take a thicker embodiment closer to the higher end of the structure range. The foam, an open cell polyurethane, is attached to the bladder layer via elastic straps. In the event of liquid spillage on the cushion, the foam may be separated from the bladder layer and cleaned or replaced separately.

### Safety Precautions

In one embodiment, the control element can operate under control of software disposed to detect and avoid states in which safety of the user is at risk. For example, if measurements returned from the transducer subsystem are ever significantly lower than the calibrated 0 psi value, or ever significantly higher than the expected max pressure experienced by the unit, the control element can determine that the associated transducer might be faulty. In such cases, faulty transducers can be ignored, and the unit may be disposed to fail softly to a safe state, such as by venting the corresponding bladder.

### DC Power Supply

The control unit is supplied with an off-the-shelf AC/DC power supply (15V 3A in the case of our implementation) that is connected in order to recharge the battery. When this power is connected, the charge chip connects the system bus to the 15V input in addition to using this input to charge the battery.

### Wireless Communications Module

The wireless communications module provides an interface that allows the microcontroller to communicate with both the internet (via a Wi-Fi connection) and the control device (via a Bluetooth connection). It is important to note that other communication methods, particularly a cellular connection, could also be used to achieve the same or possibly expanded functionality. Additionally, a simplified version of the device could be built with no wireless connectivity or less wireless connectivity. The wireless communications module serves almost entirely as a communications relay, receiving communications over bluetooth and WiFi and performing a relatively minimal amount of translation to turn them into commands that are sent to the main microcontroller over an SPI connection (other types of link between the wireless communications module and microcontroller could serve the exact same purpose). It should be noted that the wireless communications module itself contains a microcontroller, which could be used as an alternative to the microcontroller described below. Due to limitations of the interfaces available on the microcontroller in the wireless communications module, it was deemed more cost-effective to use a second microcontroller with more interfacing capability, described below, to perform the majority of the system control. Despite this, the wireless module microcontroller can be used to offload tasks from the main microcontroller. In particular, the wireless module microcontroller can be used to generate audio signals.

### Microcontroller

The microcontroller is a small microprocessor with internal code and data memory that is used for control of all of the other subsystems on the printed circuit assembly. Its most significant task is to provide control over the inflation level of the individual bladders inside the cushion. This is achieved via software containing the machine learning algorithm detailed later. Additionally, the microcontroller is connected to all of the major subsystems on the printed circuit assembly. The software running on the microcontroller provides control over power sequencing, fault detection and response and data logging. The microcontroller is connected to additional, external non-volatile memory that is used to store data logs and system firmware updates that have been received over the wireless links but not yet programmed into the storage internal to the microcontroller or wireless communication module. This non-volatile memory may also store code or data (especially audio samples) that are too large to fit into the aforementioned internal memories. In the case of our implementation, this memory is implemented as NOR flash connected over the SPI interface, but other types of memory could be used to implement similar functionality.

### Analog Front End

The analog front end provides signal conditioning to transform the signals from the pressure transducers into a form that can be accurately measured by the analog to digital converter built into the microcontroller. The signal output by the pressure transducers is a differential signal (desired quantity is encoded as the difference between two voltages) with a maximum amplitude of a small fraction of a volt, while the microcontroller is designed to measure single ended (desired quantity is encoded as the difference between a single voltage and system ground) signal with a few volts of amplitude. A high-level schematic of the Analog Front End is shown in FIG. 14. In the analog front end, all of the differential signals from the pressure transducers are first run through a pair of multiplexers (Item 1 in the figure) to select the differential signal from exactly one pressure transducer (E.g PT0+ and PT0− in the figure above for pressure transducer 0). The microcontroller software makes the microcontroller output signals (PTMUX_A,B,C in the figure) that select which transducer. Once this differential signal is selected, it is provided as in input to an instrumentation amplifier, which amplifies it (in our case by a factor of approximately 10 (See item 2 in the figure), chosen as the maximum gain that keeps the voltage output within the limitations of the amplifier for the expected range of measured pressures), and references it to a level Vref. Ideally, the instrumentation amplifier output is provided by the equation

$$Vout = ( ( Vp - Vn ) ⋆ Gain + Vref ) \qquad Eq . l :$$

where Vp and Vn are the two voltages that compose the differential signal, gain is a constant defined by the circuit design (approximately 10 in our implementation as discussed above), and Vref is a reference voltage provided to the amplifier (choice of Vref will be discussed later). Both Vref and the instrumentation amplifier output are filtered (item 4 in the figure) to remove high-frequency noise, then sampled and converted to digital by the microcontroller where a final subtraction is performed digitally to remove the effect of Vref and obtain the desired signal. The microcontroller samples all of the transducers in sequence by changing the transducer selected by the multiplexer.

The instrumentation amplifier used in the Analog Front End is a low-performance variety to enable lower overall cost of the system. With the selection of this component a significant offset in the measured differential voltage of the transducer output is introduced by nonidealities in the amplifier, in particular the input offset voltage Vos, which affects the amplifier output as shown in Eq. 2

$$Vout = ( ( Vp - Vn + Vos ) ⋆ Gain + Vref ) \qquad Eq . 2 :$$

The input offset voltage is affected by numerous parameters that vary over time, such as temperature, voltage at the amplifier inputs (this effect is also known as the common-mode rejection ratio or CMRR of the amplifier), and amplifier power supply voltage. The net effect of this is to create a time-varying offset on all pressure readings produced by the analog front end, which degrades system performance. In order to reduce this effect, Vref can also be selected by the multiplexers to be applied to both inputs of the instrumentation amplifier (Vp=Vn=Vref). According to Eq. 2, the output of the instrumentation amplifier is now Vos*Gain+Vref. When the microcontroller samples both Vout and Vref with this multiplexer selection (Input 7 in the case of the figure), it can thus assume that any difference between the two is caused by amplifier offset, and can thus trivially compute the offset of the amplifier. The microcontroller software then computes this difference and subtracts it out from any measurements taken in the future, cancelling out the error. Because the offset is time-varying, it is periodically re-measured. Finally, note that Vref is specifically selected to be very close to the common-mode voltage ((Vp+Vn)/2) output by an ideal pressure transducer reading 0 psi. This reduces the detrimental effect of the common mode rejection ratio, which would otherwise cause an unmeasured change in the offset Vos when the amplifier switched from measuring Vref to measuring the pressure transducer output with a different common-mode voltage. It is important to note that while our implementation used the same Vref as a self-calibration signal and as an output level reference for the instrumentation amplifier, this was solely done to simplify the circuit, and another voltage level could be used for the instrumentation amplifier output reference level without needing to be subject to the previously mentioned constraint to reduce the effects of CMRR. This could slightly increase the allowable gain of the amplifier before hitting voltage limitations and thereby slightly decrease the amount of noise in the measurements. In our case, the reference voltage Vref was generated using a voltage divider that divides the supply voltage to the pressure transducers by two followed by an op-amp configured as a unity-gain buffer (item 3 in the FIG. 14).

### Valve/Pump Drivers

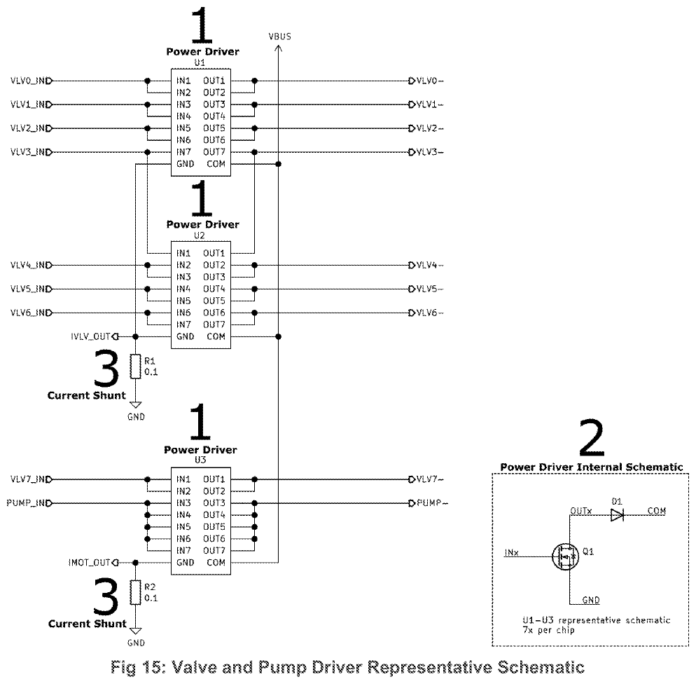

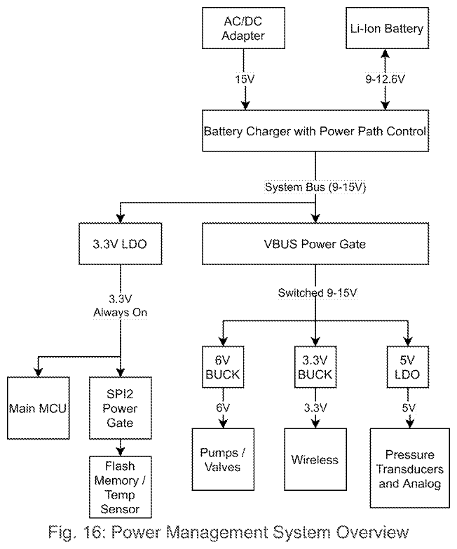

The valve/pump drivers are used to control the valves and pump in the control unit. An overview is shown in (FIG. 15). Each driver consists of a number of N-Channel MOSFET transistors in parallel (Item 1 in the figure, detailed by item 2). The number of MOSFETs in parallel is chosen to give sufficient current-handling capability. The MOSFETs switch one leg of the desired pump/valve to ground through a current shunt (Item 3 in the figure). All of the inflation valves (VLV0-VLV6) share a current shunt, and the vent valve (VLV7) and pump share another. The microcontroller monitors the voltage across these current shunts (through either or both of hardware comparators and an analog-to-digital converter) to determine valve/pump current. The other leg of the pump/valve is connected to +6V. In order to provide a current path for the stored energy in the valve/pump when the MOSFET is turned off, a diode is connected between the drain of each transistor and the main system supply rail (9-15V depending on power source). This allows the valves to be closed more quickly than returning the diode to +6V by allowing a reverse voltage to appear across the valve. The system must be designed with sufficient capacitance on the system bus so as to avoid creating an overvoltage condition when energy from the pump or valves is dumped back to the system bus upon valve closing or pump shutdown. Note that the pump may be returned to 6V instead of Vbus to avoid exacerbating this problem (not shown in figure), or all loads may be returned to +6V at the expense of slower valve closing. The solenoid valve drive transistors are fed with a pulse width modulation signal generated by the microcontroller in such a way as to minimize the power consumption of the unit. The duty cycle of the PWM signal is determined by the microcontroller software based on the system rail voltage and the time after valve opening so as to reliably open the valve using the minimum amount of power. Additionally, the measured current waveforms and bag pressures may be used to detect if the valve is open and dynamically adjust the PWM duty cycle. This allows the opportunistic use of lower duty cycles than would be possible if the opening of the solenoid could not be detected and the system were forced to use a duty cycle that would work under all conditions. The opening of the solenoid can be detected by looking for the characteristic current “dip” (Item 1 in the figure) that occurs as solenoid inductance increases due to the high-permeability actuator being sucked inside the coil. (FIG. 16).

Alternatively, pump current may be measured to see if it changes when the valve is commanded open (which would suggest a change in the head pressure of the pump corresponding to a successful valve actuation), or the pressure in the bladder may be measured to ensure that it starts rising. If the valve is not detected to open even with an increased duty cycle, the unit will alert the user of the error and may halt the pump/vent operation to prevent over/underinflation.

The duty cycle of the pump PWM is determined based on the measured current; if the current exceeds a set value the PWM output is set low for the rest of the cycle. This is used to soft-start the motor and avoid current transients that would otherwise necessitate the use of larger and more expensive power electronics. In steady-state the pump driver PWM duty cycle is 100%. The pump current limit also protects the motor driver from short-circuits, if the current limit is engaged for too long the microcontroller software shuts down the pump driver PWM entirely to prevent overheating and alerts the user of an error. When the solenoid current exceeds a setpoint (set well above the normal operating current), the solenoid driver is shut down to protect it from short circuits and the unit alerts the user of an error.

### Battery Charger

The battery charger is in charge of charging the Lithium-Ion battery (other types may also be applicable) that powers the product while it is unplugged. It consists of a DC-DC buck converter (other topologies may be applicable depending on the input power source) that converts the input from the DC power supply to the correct voltage to charge the battery according to a standard lithium ion charge cycle. A constant current is supplied until the battery reaches a specified voltage, then a constant voltage is applied until the charge terminates when the current supplied to the battery is below a charge termination threshold or the charge cycle has been in progress for longer than the maximum charge cycle length. The charge chip is capable of reducing the current into the battery when the current needed to run the system increases in order to avoid exceeding the rated output of the supplied AC/DC power supply, which must supply the combined charge and operation current. While the unit is plugged in, the battery charger connects the system bus (which powers the remainder of the system) to the DC input voltage. When the charger is not connected, the system bus rail is connected to the battery. All of the downstream circuitry must be designed to handle the abrupt change in input voltage that occurs when the charger is plugged in or unplugged. Finally, the battery charger circuit provides information to the microcontroller that is used to monitor the DC input voltage, battery voltage, input current, and battery current along with other parameters. These parameters are used to track the state of charge and state of health of the battery. Additionally, the microcontroller is able to send new charging voltage and current setpoints to the charge chip. When USB-C power delivery based charging is implemented in the future, this will enable the system to adapt its power demand to power source capabilities.

### Electrical Function

The electrical system of the control unit consists of a battery, a printed circuit assembly, a power switch, a pump (described in the section above), and solenoid valves (also described above). Additionally, a DC power supply is shipped with the unit. Of these components, only the printed circuit assembly is specifically designed for the application, although future design improvements may include development of customized versions of the aforementioned components. A block diagram of the control unit electronics is provided in (FIG. 13).

### Printed Circuit Assembly

The printed circuit assembly (FIG. 8, 003) consists of the following major subsystems: A battery charger, a wireless communications module, a microcontroller, a number of pressure transducers, an analog front end, a number of pump/solenoid valve drivers, an audio amplifier, and a power management system.

### Pressure Transducers

The system contains, at a minimum, one pressure transducer per inflatable bladder that is used to measure bladder pressure and feed the control loop. In our system, these are piezoresistive MEMS pressure transducers. They are manufactured as a bridge circuit made from strain sensitive resistors and output a differential voltage that is fed into the analog front end described below before finally being measured by the microcontroller. Lastly, a digital temperature sensor is optionally placed near the pressure transducers to allow software to compensate for error in the pressure readings caused by temperature variations. In order to aid in this, the pressure transducers may be placed in a thermal “island” on the printed circuit assembly that is isolated from the rest of the board by routed out slots in order to make all of the pressure transducers operate as close to the same temperature as possible.

It is worth noting that other types of pressure transducers could be used with no effect on functionality. Additionally, fewer pressure transducers could be used along with valves to select the pressure being sampled, or a singular pressure transducer could be placed on the pump/valve line to reuse the existing valves for this task. This would result in an acoustically noisier, more power hungry system with a much slower pressure response, and thus was not selected for our design.

### Power Management Subsystem

The control unit printed circuit assembly contains a power management subsystem to allow the unit to achieve the following key functions in addition to the power related functions mentioned previously: turning the unit on and off, gating power to unneeded parts of the unit to extend battery life, and converting power to the appropriate voltage for other subsystems. In future units, this may also interface with a USB type C connection to provide USB Power Delivery functionality and allow battery charging over USB. A high-level diagram of the Power Management Subsystem is shown in (FIG. 17).

### Turning the Unit on and Off:

In normal operation, the control unit printed circuit assembly is never truly “off” after the battery is first installed at the factory. When the user presses the power button to turn off the unit, the main microcontroller safely shuts down all of the subsystems which can be power gated and then removes their power, then turns off the power light. The system is also powered off when the battery voltage goes below a preset threshold. Only the battery charge controller (which may be put into a low-power mode) and the main microcontroller (which is also put into a low-power mode) continue to receive power in the fully power-gated system. In this state even a battery with only 10% charge remaining can last for thousands of hours. In this “off” mode, the microcontroller may be woken up (and the system powered on) by the power switch. Additionally, the microcontroller may be woken up periodically and power to subsystems temporarily reapplied to enable the unit to be powered on by a change in bladder pressures, time based alarm, bluetooth, WiFi, or other external stimulus. Whenever the microcontroller software resumes normal operation the power light is turned back on.

The unit has several power switches that can be controlled by the microcontroller software to minimize power consumption. These are as follows. It is worth noting that no power gating is required to create a functional device, it is used to improve battery life and may also be used as a method to shut down power to components that the software has determined to be faulty in order to reduce the risk of harm to the user.
SPI2 power gate: A P-Channel MOSFET used to remove 3.3V power to the flash memory and pressure transducer temperature sensor.
VBUS power gate: A slew-rate controlled N-Channel MOSFET that removes power to the 6V converter that powers the pumps and solenoids, the 3.3V converter that powers the wireless module, the 5V regulator that powers the pressure transducers, and the system voltage connection to the valve/pump drivers.
3.3V/6V converter power off: The 3.3V (wireless module) and 6V (pump/valves) buck converters have their own individual shutdown inputs controlled by the microcontroller. These have more leakage than the VBUS power gate but are also more granular.
Chips with Low-Power modes: The wireless module, main microcontroller, and battery charger all have vendor-supplied low power modes that can be activated through software.

### Power Conversion:

The system has a total of 5 major power rails that are not generated internally to the component they feed. They are as follows:
System Bus: This rail is between 9-15V DC. It is controlled by the battery charger, which selects the highest of the battery voltage and the system DC power input. Everything in the system, including the battery charger itself, is powered either directly or indirectly from this rail.
3.3V MCU: Generated by a low-quiescent-current linear regulator, this power rail supplies the main microcontroller, external flash memory attached to the microcontroller, and the pressure transducer temperature sensor (if present). This regulator cannot be shut down unless the unit is unplugged and the battery is so exhausted that it is disconnected by its internal protection circuit, a case that should never happen in normal operation (the power gating should dramatically reduce power consumption upon detection of low battery voltage prior to this point to prevent further battery discharge). It is worth noting that the system can recover from a loss of battery voltage due to protection circuit shutdown when DC power input is re-applied.
5V Analog: This rail powers the pressure transducers and analog front end. It is generated by a linear regulator. The input to this linear regulator is gated by the VBUS power gate.
3.3V Wireless: This rail exclusively feeds the wireless module. It is generated by a 3.3V DC-DC buck converter from the system bus. The input to this regulator is gated by the VBUS power gate. The regulator additionally has its own individual shutdown control.
6V: This rail feeds the pump and valves. It is generated by a 6V DC-DC buck converter from the system bus. The input to this regulator is gated by the VBUS power gate. The regulator additionally has its own individual shutdown control.

### Magnetic Mounting

Magnets are installed into the base of the control box (FIG. 7, 004). This serves to facilitate by the user the ability to replace the mount accessory utilized for securing the control box to a structure. Separate mount accessories, capable of securing to structures such as wheelchair structural tubing or headrests, are magnetically attracted to the box, and via a mechanical protrusion in the mount base secondarily retained from sliding along the box base.

### Unit Health Monitoring

The pressure cushion system can also be disposed to log one or more parameters so as to aid in manufacturer diagnostics and technical support. The logged parameters may be transmitted to the manufacturer either by having the system directly connected to the Internet or by storing them in volatile or nonvolatile memory to be transmitted to the internet using an external control device (such as described herein). Parameters that may be monitored include, but are not limited to: battery voltage, battery cycle count, time the pump has been in operation, number of solenoid or pump cycles, solenoid drive current waveforms, solenoid duty cycles, temperature of various components, specifically including; the pressure transducers or the battery, firmware and App versions, any detected software exceptions, auto-calibration results and leak rates, control system coefficients (as described with respect to the section on Machine Learning), serial number, connected Wi-Fi and Bluetooth device network names and addresses, and system pressures. These parameters may also be processed remotely by related IT infrastructure and used to inform the user and/or one or more caretakers with respect to system performance.

### Audio Amplifier

The printed circuit assembly may contain a Class-D (switching) integrated audio amplifier in order to allow it to produce pleasing tones and spoken words to inform the user of system status or provide reminders to the user. In the case of this specific implementation, the audio may be generated by the wireless microcontroller instead of the main microcontroller in order to more effectively use the peripherals on each microcontroller and distribute CPU load. The playback of the audio samples is commanded using the same communication link used to send translated commands and data between the wireless module and main microcontroller. Alternative types of audio amplifiers may also be used, as well as different sources of audio, with little to no functional impact.

Communicating with Users

In certain states it may be necessary to inform the user of the cushion of a state or change of state in the cushion. This communication may occur with either the individual sitting on the cushion or with one of the individuals who are responsible for the wellbeing of the individual who is sitting on the cushion. Communication may be used in, although is not limited to, the following ways: an audible notification, either from the control box or from a mobile control unit, shifting pressure between the bladders, submitting a push notification to the user's mobile control unit, a light on the control box.

### Calibration

Due to variances in the pressure transducers and the electronic components used to read from them, it may be necessary to automatically calibrate the control system to compensate for these variances. The calibration procedure may take one of many forms, but here is one such form.

First, the offset of each transducer may be determined by completely venting the zone and attached pneumatics that map to the transducer. After waiting for a time to allow the pressure to equalize, the value of the transducer may be recorded and used as a baseline offset. The baseline can now be subtracted from the raw value returned by the transducer in operation to gain a more accurate measurement.

Second, to calibrate against transducer-to-transducer variation, each transducer's operating range may be compared against one ground-truth transducer. This will not improve the absolute accuracy of any given transducer, but will ensure that all transducers on the unit have similar scaling. This calibration scenario may be realized by activating multiple solenoids simultaneously and allowing their pneumatic regions to equalize, while using the pump and/or vent to direct the pressure up and down. The pressure relationships between the calibrating transducer and the one ground-trute transducer will be recorded, and can be consulted whenever the transducer in question is read from.

### Other Monitoring Options

There are a number of alternative uses for the cushion and/or support. For example, the cushion and/or support can be disposed for use by medical patients who are relatively immobile, either because their musculature is weakened, because they are restrained by medical devices, or because medical personnel would like them not to move selected parts of their bodies (whether because those selected parts are healing, because movement would disrupt or interfere with medical devices or with user healing, or because movement would cause undue pain to the user). For another example, the cushion and/or support can be disposed for use by persons who are confined for relatively long periods of time, such as drivers engaged in automobile travel, pilots or other personnel (such as medical or military personnel) who are engaged in activity that demands their full attention and for which they cannot move about. For another example, the cushion and/or support can be disposed for use by persons who are required by their activity to position themselves in an awkward or strained manner.

Users may read any of the below measurements via the user's mobile control unit. Users may also set desired thresholds for this information, and be alerted using one of the methods described below if any measurements exceed the provided thresholds:
the position of the unit;
the orientation of the unit, such as whether the unit is parallel to the ground or is tilted, the latter possibly indicating that the user has tilted a wheelchair on which the cushion is resting;
the temperature of the user;
the temperature of the ambient environment;
the moisture within the cushion component of the unit;
the state of charge of a battery associated the unit, or the state of another power source associated with the unit.

The position of the unit can be estimated with a variety of methods, including GPS, Cellular positioning, adjacency to various wifi devices, and adjacency to various bluetooth devices. The temperature of the user, the atmosphere, and/or moisture within the cushion component of the unit may all be measured directly with various sensors.

1-96. (canceled)

97. A cushion system comprising:
a cushion component including a plurality of bladders;
a control component including a pneumatic subsystem and an electrical subsystem, the pneumatic subsystem being pneumatically coupled to the plurality of bladders and including a bladder-specific pressure transducer for each bladder of the plurality of bladders; and
pneumatic lines including, a bladder-specific pneumatic line that pneumatically couples a bladder of the plurality of bladders to a corresponding bladder-specific pressure transducer;
wherein the electrical subsystem is configured to predict an actual pressure within the bladder from a measured pressure that is generated in response to a signal received from the bladder-specific pressure transducer that corresponds to the bladder.

98. The cushion system of claim 97, wherein the electrical subsystem includes a predictor system configured within a microcontroller of the electrical subsystem, the predictor system configured to predict the actual pressure within the bladder in response to receiving 1) a measured pressure at a time a prediction request is made, 2) a measured pressure prior to a start of a current pump or vent operation, and 3) how much time has elapsed between the start of the current pump or vent operation and the time the prediction request was made.

99. The cushion system of claim 98, wherein the predictor system includes a bladder-specific set of weights for each bladder of the plurality of bladders.

100. The cushion system of claim 99, wherein the bladder-specific set of weights for each bladder of the plurality of bladders includes two separate sets of weights, one set of weights for use in a pump operation, and one set of weights for use in a vent operation.

101. The cushion system of claim 97, wherein the electrical subsystem includes a predictor system configured within a microcontroller of the electrical subsystem, the predictor system configured to implement a polynomial function to predict the actual pressure from the measured pressure.

102. The cushion system of claim 101, wherein the predictor system is configured to determine a set of coefficients of the polynomial function in response to feedback from an external signal.

103. A method comprising:
receiving a signal from a bladder-specific pressure transducer that is pneumatically coupled to a bladder of a cushion system that includes a plurality of bladders and a plurality of bladder-specific pressure transducers, wherein the bladder-specific pressure transducer is pneumatically coupled to the bladder via a bladder-specific pneumatic line;
generating a measured pressure from the received signal; and
predicting an actual pressure within the bladder in response to the measured pressure.

104. The method of claim 103, wherein the electrical subsystem includes a predictor system configured within a microcontroller of the electrical subsystem, the predictor system configured to predict the actual pressure within the bladder in response to receiving 1) a measured pressure at a time a prediction request is made, 2) a measured pressure prior to a start of a current pump or vent operation, and 3) how much time has elapsed between the start of the current pump or vent operation and the time the prediction request was made.

105. The method of claim 104, wherein the predictor system includes a bladder-specific set of weights for each bladder of the plurality of bladders.

106. The method of claim 105, wherein the bladder-specific set of weights for each bladder of the plurality of bladders includes two separate sets of weights, one set of weights for use in a pump operation, and one set of weights for use in a vent operation.

107. The method of claim 103, wherein predicting an actual pressure within the bladder in response to the measured pressure involves applying a machine learning technique to a sequence of pressure measurements associated with the bladder.

108. The method of claim 103, wherein predicting an actual pressure within the bladder in response to the measured pressure involves implementing a polynomial function to predict the actual pressure from the measured pressure.

109. The method of claim 108, wherein predicting an actual pressure within the bladder in response to the measured pressure involves determining a set of coefficients of the polynomial function in response to feedback from an external signal.

110. A control component for a cushion system, the control component comprising:
a pneumatic subsystem; and
an electrical subsystem;
the pneumatic subsystem including couplers configured to provide a connection to pneumatic lines that pneumatically connect a plurality of bladders of a cushion component of the cushion system to the pneumatic subsystem, and including a bladder-specific pressure transducer for each bladder of the plurality of bladders;
wherein the electrical subsystem is configured to predict an actual pressure within a bladder from a measured pressure that is generated in response to a signal received from a bladder-specific pressure transducer that corresponds to the bladder.

111. The control component of claim 110, wherein the electrical subsystem includes a predictor system configured within a microcontroller of the electrical subsystem, the predictor system configured to predict the actual pressure within the bladder in response to receiving 1) a measured pressure at a time a prediction request is made, 2) a measured pressure prior to a start of a current pump or vent operation, and 3) how much time has elapsed between the start of the current pump or vent operation and the time the prediction request was made.

112. The control component of claim 111, wherein the predictor system includes a bladder-specific set of weights for each bladder of the plurality of bladders.

113. The control component of claim 112, wherein the bladder-specific set of weights for each bladder of the plurality of bladders includes two separate sets of weights, one set of weights for use in a pump operation, and one set of weights for use in a vent operation.

114. The control component of claim 110, wherein the electrical subsystem includes a predictor system configured within a microcontroller of the electrical subsystem, the predictor system configured to implement a polynomial function to predict the actual pressure from the measured pressure.

115. The control component of claim 114, wherein the predictor system is configured to determine a set of coefficients of the polynomial function in response to feedback from an external signal.
